(19) United States
(12) Patent Application Publication
(10) Pub. No.: US 2026/0297059 A1
(43) Pub. Date: Oct. 1, 2026

(54) CRYSTAL FORMS OF ACEQUINOCYL, AND THEIR PREPARATION METHODS AND APPLICATIONS

(71) Applicant: YONGNONG BIOSCIENCES CO., LTD., Shaoxing (CN)

(72) Inventors: Chuan REN, Shaoxing (CN); Chengjun WU, Shaoxing (CN); Chenlei MAO, Shaoxing (CN); Haifeng HU, Shaoxing (CN); Zijie SONG, Shaoxing (CN); Xin QIN, Shaoxing (CN); Dengke ZHANG, Shaoxing (CN); Xinhao FU, Shaoxing (CN); Chunhui MAO, Shaoxing (CN)

(73) Assignee: YONGNONG BIOSCIENCES CO., LTD., Shaoxing (CN)

(21) Appl. No.: 19/540,681

(22) Filed: Feb. 14, 2026

(30) Foreign Application Priority Data
Mar. 27, 2025 (CN) 202510376996.4

(51) Int. Cl. C07D 401/14; A61K 31/519

(52) U.S. Cl. CPC C07D 401/14; A61K 31/519; C07B 2200/13

(57) ABSTRACT

Embodiments of the present disclosure provide a crystal form of acequinocyl represented by formula (I), and its preparation method and application. Formula (I) is

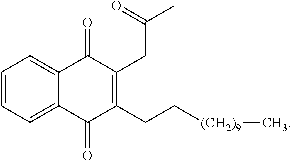

An X-ray powder diffraction pattern (Cu-Kα radiation) of the crystal form has a characteristic diffraction peak at 3.3° 2θ±0.2° 2θ and a characteristic diffraction peak(s) at at least one of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ.

### CROSS-REFERENCE TO RELATED APPLICATIONS

This application claims priority to the Chinese Patent Application No. 202510376996.4, filed on Mar. 27, 2025, the contents of which are hereby incorporated by reference.

### TECHNICAL FIELD

The present disclosure relates to the field of acaricides, and in particular, to crystal forms of acequinocyl, and their preparation methods and applications.

### BACKGROUND

Acequinocyl (2-(acetyloxy)-3-dodecyl-1,4-naphthoquinone) is widely used worldwide for mite control in agricultural, household, and other related fields due to its characteristics of high efficiency, low toxicity, broad spectrum, and biodegradability. However, in the preparation of pesticide formulations, especially aqueous suspension concentrates, the commercially available crystal forms of acequinocyl exhibit poor system compatibility and stability and are highly prone to creaming, solidification, and other adverse phenomena. This leads to severe product deterioration and failure to be used normally. The aforesaid problems have greatly limited the application of acequinocyl aqueous suspension concentrates.

Therefore, it is necessary to provide a novel crystal form of acequinocyl and a method for preparing the crystal form to solve the aforesaid problems.

### SUMMARY

One or more embodiments of the present disclosure provide a crystal form of acequinocyl represented by formula (I):

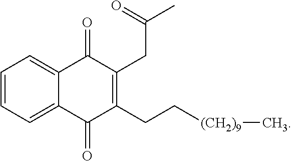

An X-ray powder diffraction pattern (Cu-Kα radiation) of the crystal form has a characteristic diffraction peak at 3.3° 2θ±0.2° 2θ and a characteristic diffraction peak(s) at at least one of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ.

In some embodiments, the X-ray powder diffraction pattern (Cu-Kα radiation) of the crystal form has the characteristic diffraction peak at 3.3° 2θ±0.2° 2θ and characteristic diffraction peaks at at least two of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ.

In some embodiments, the X-ray powder diffraction pattern (Cu-Kα radiation) of the crystal form has the characteristic diffraction peak at 3.3° 2θ±0.2° 2θ and characteristic diffraction peaks at at least three of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ.

In some embodiments, the X-ray powder diffraction pattern (Cu-Kα radiation) of the crystal form has the characteristic diffraction peak at 3.3° 2θ±0.2° 2θ and characteristic diffraction peaks at at least five of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ.

In some embodiments, the X-ray powder diffraction pattern (Cu-Kα radiation) of the crystal form has the characteristic diffraction peak at 3.3° 2θ±0.2° 2θ and characteristic diffraction peaks at at least seven of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ.

In some embodiments, the X-ray powder diffraction pattern (Cu-Kα radiation) of the crystal form has characteristic diffraction peaks at 3.3° 2θ±0.2° 2θ, 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, and 24.5° 2θ±0.2° 2θ.

In some embodiments, a relative intensity of the characteristic diffraction peak at 3.3° 2θ±0.2° 2θ is defined as 100%, and relative intensities of characteristic diffraction peaks at 10.1° 2θ±0.2° 2θ and 10.4° 2θ±0.2° 2θ are greater than 5%.

In some embodiments, the relative intensity of the characteristic diffraction peak at 3.3° 2θ±0.2° 2θ is defined as 100%, and relative intensities of characteristic diffraction peaks at 11.0° 2θ±0.2° 2θ, 13.5° 2θ±0.2° 2θ, and 22.1° 2θ±0.2° 2θ are greater than 3%.

In some embodiments, the relative intensity of the characteristic diffraction peak at 3.3° 2θ±0.2° 2θ is defined as 100%, and relative intensities of characteristic diffraction peaks at 6.7° 2θ±0.2° 2θ, 14.6° 2θ±0.2° 2θ, 17.1° 2θ±0.2° 2θ, and 24.5° 2θ±0.2° 2θ are greater than 1%.

In some embodiments, the X-ray powder diffraction pattern (Cu-Kα radiation) of the crystal form has characteristic diffraction peaks at 3.3465° 2θ±0.2° 2θ, 6.7245° 2θ±0.2° 2θ, 10.1118° 2θ±0.2° 2θ, 10.4289° 2θ±0.2° 2θ, 10.9607° 2θ±0.2° 2θ, 13.5235° 2θ±0.2° 2θ, 14.5662° 2θ±0.2° 2θ, 17.1189° 2θ±0.2° 2θ, 22.0684° 2θ±0.2° 2θ, and 24.5351° 2θ±0.2° 2θ.

In some embodiments, the X-ray powder diffraction pattern (Cu-Kα radiation) of the crystal form has characteristic diffraction peaks at 3.3513° 2θ±0.2° 2θ, 6.7269° 2θ±0.2° 2θ, 10.1144° 2θ±0.2° 2θ, 10.4262° 2θ±0.2° 2θ, 10.9626° 2θ±0.2° 2θ, 13.5256° 2θ±0.2° 2θ, 14.5699° 2θ±0.2° 2θ, 17.1275° 2θ±0.2° 2θ, 22.0641° 2θ±0.2° 2θ, and 24.5299° 2θ±0.2° 2θ.

In some embodiments, the X-ray powder diffraction pattern (Cu-Kα radiation) of the crystal form has characteristic diffraction peaks at 3.3874° 2θ±0.2° 2θ, 6.7684° 2θ±0.2° 2θ, 10.1553° 2θ±0.2° 2θ, 10.4614° 2θ±0.2° 2θ, 10.9971° 2θ±0.2° 2θ, 13.5633° 2θ±0.2° 2θ, 14.6231° 2θ±0.2° 2θ, 17.1483° 2θ±0.2° 2θ, 22.0848° 2θ±0.2° 2θ, and 24.5728° 2θ±0.2° 2θ.

In some embodiments, the X-ray powder diffraction pattern (Cu-Kα radiation) of the crystal form has characteristic diffraction peaks at 3.3794° 2θ±0.2° 2θ, 6.7564° 2θ±0.2° 2θ, 10.1493° 2θ±0.2° 2θ, 10.4483° 2θ±0.2° 2θ, 10.9867° 2θ±0.2° 2θ, 13.5568° 2θ±0.2° 2θ, 14.5973° 2θ±0.2° 2θ, 17.1544° 2θ±0.2° 2θ, 22.0885° 2θ±0.2° 2θ, and 24.5680° 2θ±0.2° 2θ.

In some embodiments, a relative intensity of the characteristic diffraction peak at 3.3465° 2θ±0.2° 2θ is defined as 100%, and relative intensities of the characteristic diffraction peaks at 6.7245° 2θ±0.2° 2θ, 10.1118° 2θ±0.2° 2θ, 10.4289° 2θ±0.2° 2θ, 10.9607° 2θ±0.2° 2θ, 13.5235° 2θ±0.2° 2θ, 14.5662° 2θ±0.2° 2θ, 17.1189° 2θ±0.2° 2θ, 22.0684° 2θ±0.2° 2θ, and 24.5351° 2θ±0.2° 2θ are 1.88%, 8%, 10.52%, 6.65%, 6.47%, 1.72%, 2.86%, 5.9%, and 2.56%, respectively.

In some embodiments, a relative intensity of the characteristic diffraction peak at 3.3513° 2θ±0.2° 2θ is defined as 100%, and relative intensities of the characteristic diffraction peaks at 6.7269° 2θ±0.2° 2θ, 10.1144° 2θ±0.2° 2θ, 10.4262° 2θ±0.2° 2θ, 10.9626° 2θ±0.2° 2θ, 13.5256° 2θ±0.2° 2θ, 14.5699° 2θ±0.2° 2θ, 17.1275° 2θ±0.2° 2θ, 22.0641° 2θ±0.2° 2θ, and 24.5299° 2θ±0.2° 2θ are 1.77%, 8.54%, 16.7%, 11.03%, 5.65%, 2.09%, 3.36%, 10.19%, and 3.73%, respectively.

In some embodiments, a relative intensity of the characteristic diffraction peak at 3.3874° 2θ±0.2° 2θ is defined as 100%, and relative intensities of the characteristic diffraction peaks at 6.7684° 2θ±0.2° 2θ, 10.1553° 2θ±0.2° 2θ, 10.4614° 2θ±0.2° 2θ, 10.9971° 2θ±0.2° 2θ, 13.5633° 2θ±0.2° 2θ, 14.6231° 2θ±0.2° 2θ, 17.1483° 2θ±0.2° 2θ, 22.0848° 2θ±0.2° 2θ, and 24.5728° 2θ±0.2° 2θ are 2.04%, 7.03%, 9.41%, 5.88%, 6.25%, 1.58%, 2.13%, 4.65%, and 1.55%, respectively.

In some embodiments, a relative intensity of the characteristic diffraction peak at 3.3794° 2θ±0.2° 2θ is defined as 100%, and relative intensities of the characteristic diffraction peaks at 6.7564° 2θ±0.2° 2θ, 10.1493° 2θ±0.2° 2θ, 10.4483° 2θ±0.2° 2θ, 10.9867° 2θ±0.2° 2θ, 13.5568° 2θ±0.2° 2θ, 14.5973° 2θ±0.2° 2θ, 17.1544° 2θ±0.2° 2θ, 22.0885° 2θ±0.2° 2θ, and 24.5680° 2θ±0.2° 2θ are 1.5%, 6.18%, 5.62%, 3.81%, 4.67%, 1.2%, 1.99%, 3.49%, and 2.74%, respectively.

In some embodiments, the X-ray powder diffraction pattern of the crystal form is substantially as depicted in FIG. 1A, FIG. 2A, FIG. 3A, or FIG. 4A. For example, the X-ray powder diffraction pattern of the crystal form is substantially as depicted in FIG. 1A.

In some embodiments, a differential scanning calorimetry thermogram of the crystal form, obtained at a heating rate of 5° C./min, has at least one characteristic thermal event in a range of 55° C. to 60° C.

In some embodiments, the differential scanning calorimetry thermogram of the crystal form, obtained at the heating rate of 5° C./min, has at least one characteristic thermal event in a range of 57° C. to 58° C.

In some embodiments, the differential scanning calorimetry thermogram of the crystal form, obtained at the heating rate of 5° C./min, has at least one characteristic thermal event in a range of 58° C. to 59° C.

In some embodiments, the differential scanning calorimetry thermogram of the crystal form, obtained at the heating rate of 5° C./min, has at least one characteristic thermal event in a range of 57.03° C. to 57.81° C.

In some embodiments, the differential scanning calorimetry thermogram of the crystal form, obtained at the heating rate of 5° C./min, has at least one characteristic thermal event in a range of 56.96° C. to 57.55° C.

In some embodiments, the differential scanning calorimetry thermogram of the crystal form, obtained at the heating rate of 5° C./min, has at least one characteristic thermal event in a range of 57.84° C. to 58.51° C.

In some embodiments, the differential scanning calorimetry thermogram of the crystal form, obtained at the heating rate of 5° C./min, has at least one characteristic thermal event in a range of 57.32° C. to 57.58° C.

In some embodiments, the differential scanning calorimetry thermogram of the crystal form is substantially as depicted in FIG. 1B, FIG. 2B, FIG. 3B, or FIG. 4B. For example, the differential scanning calorimetry thermogram of the crystal form is substantially as depicted in FIG. 1B.

In some embodiments, an aqueous suspension concentrate prepared from the crystal form has a particle size increase of less than 300% after thermal storage at a temperature greater than 50° C. for over 10 days and subsequent storage at room temperature for not less than 30 days.

One or more embodiments of the present disclosure provide a method for preparing the crystal form according to the embodiments of the present disclosure. The method includes a first method, a second method, or a third method.

In some embodiments, the first method includes dissolving the acequinocyl represented by formula (I) in a crystallization solvent, heating to 55° C. to 70° C., cooling to crystallize, and filtering to obtain the crystal form. In some embodiments, in the first method, a weight ratio of the crystallization solvent to the acequinocyl represented by formula (I) is (0.5-10):1. In some embodiments, in the first method, a crystallization temperature of the acequinocyl represented by formula (I) is in a range of 0° C. to 70° C., for example, 5° C., 10° C., 15° C., 20° C., 25° C., 30° C., 35° C., 40° C., 45° C., 50° C., 55° C., 60° C., or 65° C., preferably 5° C. to 20° C. In some embodiments, in the first method, the crystallization solvent includes at least one of water, alcohol, ether, or hydrocarbon. The alcohol includes at least one of methanol, ethanol, or isopropanol. The ether includes 2-methyltetrahydrofuran. The hydrocarbon includes at least one of n-hexane, cyclohexane, n-pentane, or n-heptane. Preferably, the crystallization solvent includes at least one of methanol, ethanol, or 2-methyltetrahydrofuran.

In some embodiments, the second method includes heating the acequinocyl represented by formula (I) to 55° C. to 70° C. to melt, cooling and slicing to obtain the crystal form. In some embodiments, in the second method, a slicing temperature of the acequinocyl represented by formula (I) is in a range of 0° C. to 70° C., for example, 5° C., 10° C., 15° C., 20° C., 25° C., 30° C., 35° C., 40° C., 45° C., 50° C., 55° C., 60° C., or 65° C., preferably 5° C. to 20° C.

In some embodiments, a third method includes heating the acequinocyl represented by formula (I) to 55° C. to 70° C. to melt, cooling to crystallize, and filtering to obtain the crystal form. In some embodiments, in the third method, a crystallization temperature of the acequinocyl represented by formula (I) is in a range of 0° C. to 70° C., for example, 5° C., 10° C., 15° C., 20° C., 25° C., 30° C., 35° C., 40° C., 45° C., 50° C., 55° C., 60° C., or 65° C., preferably 5° C. to 20° C.

One or more embodiments of the present disclosure provide an agrochemical composition. The agrochemical composition includes the crystal form according to the embodiments of the present disclosure and one or more excipients or additives.

One or more embodiments of the present disclosure provide a method for controlling agricultural pests. The method includes subjecting the agricultural pests or a region in which the agricultural pests reside to the crystal form according to the embodiments of the present disclosure, and/or to the agrochemical composition according to the embodiments of the present disclosure.

### BRIEF DESCRIPTION OF THE DRAWINGS

The present disclosure is further describable in terms of exemplary embodiments.

These exemplary embodiments are describable in detail with reference to the drawings. These embodiments are non-limiting exemplary embodiments, in which like reference numerals represent similar structures throughout the several views of the drawings, and wherein:

FIG. 1A is an X-ray powder diffraction pattern of a crystal form of acequinocyl prepared in Embodiment 1 of the present disclosure;

FIG. 1B is a differential scanning calorimetry thermogram of the crystal form of acequinocyl prepared in Embodiment 1 of the present disclosure;

FIG. 2A is an X-ray powder diffraction pattern of a crystal form of acequinocyl prepared in Embodiment 2 of the present disclosure;

FIG. 2B is a differential scanning calorimetry thermogram of the crystal form of acequinocyl prepared in Embodiment 2 of the present disclosure;

FIG. 3A is an X-ray powder diffraction pattern of a crystal form of acequinocyl prepared in Embodiment 3 of the present disclosure;

FIG. 3B is a differential scanning calorimetry thermogram of the crystal form of acequinocyl prepared in Embodiment 3 of the present disclosure;

FIG. 4A is an X-ray powder diffraction pattern of a crystal form of acequinocyl prepared in Embodiment 4 of the present disclosure;

FIG. 4B is a differential scanning calorimetry thermogram of the crystal form of acequinocyl prepared in Embodiment 4 of the present disclosure;

FIG. 5A is an X-ray powder diffraction pattern of a crystal form of acequinocyl in Comparative Example 1 of the present disclosure;

FIG. 5B is a differential scanning calorimetry thermogram of the crystal form of acequinocyl in Comparative Example 1 of the present disclosure;

FIG. 6 is a photograph illustrating stability of Comparative Example 1 of the present disclosure after thermal storage followed by storage at room temperature for 2 hours;

FIG. 7A is a photograph illustrating stability of the crystal form of acequinocyl of Embodiment 1 of the present disclosure after thermal storage followed by storage at room temperature for 2 hours;

FIG. 7B is a photograph illustrating stability of the crystal form of acequinocyl of Embodiment 1 of the present disclosure after thermal storage followed by storage at room temperature for 14 days;

FIG. 8A is a photograph illustrating stability of the crystal form of acequinocyl of Embodiment 1 of the present disclosure after thermal storage followed by storage at room temperature for 1 month;

FIG. 8B is a photograph illustrating stability of the crystal form of acequinocyl of Embodiment 2 of the present disclosure after thermal storage followed by storage at room temperature for 1 month;

FIG. 8C is a photograph illustrating stability of the crystal form of acequinocyl of Embodiment 3 of the present disclosure after thermal storage followed by storage at room temperature for 1 month; and

FIG. 8D is a photograph illustrating stability of the crystal form of acequinocyl of Embodiment 4 of the present disclosure after thermal storage followed by storage at room temperature for 1 month.

### DETAILED DESCRIPTION

The terminology used herein is for the purpose of describing particular embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in the present disclosure, specify the presence of stated features, integers, steps, operations, elements, and/or components, but do not preclude the presence or addition of one or more other features, integers, steps, operations, elements, components, and/or groups thereof.

Numerical ranges recited herein are intended to concisely denote each numerical value encompassed within the range.

Acequinocyl (2-(acetyloxy)-3-dodecyl-1,4-naphthoquinone) is a naphthoquinone derivative and a contact acaricide that is effective against lice, mites, and other invertebrates. Owing to its lack of systemic activity, it is widely used for the control of mite infestations on fruit trees (e.g., pear, peach, citrus, etc.) and vegetables (e.g., tomato, eggplant, pepper, etc.).

At room temperature, acequinocyl is a pale yellow powdery solid with a relatively low melting point and good stability. It is poorly soluble in water and much more soluble in organic solvents (e.g., toluene, dichloromethane, ethyl acetate, etc.).

Current pesticide formulations include suspension concentrate (SC) formulations (also referred to as suspension agents, aqueous suspension concentrates, etc.) and emulsion concentrate (EC) formulations (also referred to as suspoemulsions, etc.). As two common formulation dispersion systems, they play an important role in the pesticide field. At equivalent efficacy, the SC formulations exhibit lower phytotoxicity than the EC formulations. Therefore, from the perspective of health and environmental protection, pesticide formulations are preferably formulated as the SC formulations. However, if a solid dispersed phase in an SC formulation exists in an unstable form, polymorphic transformation may occur, leading to crystal particle size growth. This causes the formulation to develop issues such as increased viscosity and potential solidification (where solid particles in a suspension gradually aggregate and tend to form a solid under specific conditions), which may in turn clog application components (e.g., spray nozzles of pesticide sprayers) of agricultural application equipment (e.g., pesticide sprayers for fertilization, seeding, irrigation, spraying and other agricultural operations) during the spraying of pesticide formulations.

Understandably, due to its relatively low melting point, acequinocyl is prone to particle size growth, creaming, and agglomeration during the processing and heating of the suspension concentrates. It exhibits poor stability and is susceptible to product deterioration, which greatly limits the application of acequinocyl in suspension concentrate formulations. In addition, technical grade acequinocyl exhibits a low initial melting temperature, a wide melting range, and poor crystal form stability. These characteristics are also regarded as key reasons for the tendency toward creaming and poor fluidity during the preparation of acequinocyl suspension concentrates.

Typically, the stability of the acequinocyl suspension concentrates is enhanced by modifying adjuvant systems or adding special adjuvants. It should be noted, however, that the special adjuvants often give rise to other problems such as increased production costs and higher production requirements.

In the embodiments of the present disclosure, a specific crystal form of acequinocyl is used to prepare suspension concentrates. The specific crystal form has a higher melting point, a higher initial melting temperature, and a narrower melting range, thereby yielding acequinocyl suspension concentrates (i.e., an agrochemical composition described hereinafter) with enhanced stability and a reduced tendency for creaming and other related issues. Compared with the preparation of acequinocyl suspension concentrates via the special adjuvants, preparing such suspension concentrates using the specific crystal form of acequinocyl in the embodiments of the present disclosure features lower production costs and simpler operations, and can be more broadly applied in the control of agricultural pests.

Embodiments of the present disclosure provide a crystal form of acequinocyl represented by formula (I). The formula (I) (i.e., structural formula) of acequinocyl may be represented as

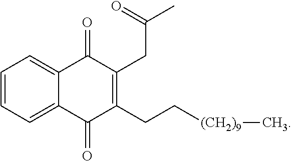

Molecular formula of acequinocyl represented by formula (I) is C₂₄H₃₂O₄ with a relative molecular weight of 384.51, and its Chemical Abstracts Service Registry Number (CAS number) is 57960-19-7.

During the crystallization process of a substance, different intramolecular or intermolecular bonding modes result in different arrangements of molecules or atoms in the crystal lattice, thereby forming distinct crystal structures referred to as different crystal forms. For same substance with different crystal forms, even if its constituent elements are identical, its physical, chemical, and biological properties differ due to the different crystal forms.

Methods for detecting crystal forms fall into two categories: physical methods and chemical methods. Physical methods utilize differences in physical properties of a substance for crystal form detection, with common approaches including X-ray diffraction (XRD) and thermal analysis. Chemical methods rely on differences in chemical properties, among which the solubility method is widely used (solubility refers to the concentration of a substance in a solvent at saturation under specific temperature and pressure, and different crystal forms have different solubilities, the crystal form of a substance can be determined by measuring its solubility), etc.

The XRD is a method that uses a diffraction phenomenon of X-rays in crystals to study a phase and crystal structure of a substance. When the X-rays are projected onto a crystal, atoms in the crystal scatter the X-rays. Because atoms are arranged periodically in the crystal, interference occurs between scattered waves, resulting in mutual reinforcement of the scattered waves in certain directions and forming diffraction phenomena to obtain a diffraction pattern. The distribution and intensity of the diffraction pattern are closely related to a crystal structure of the crystal. Therefore, by analyzing the diffraction pattern, phase and crystal structure of the substance can be determined. The XRD has advantages such as fast detection speed, high precision, and a wide application range, and is one of the important methods for crystal form detection.

The XRD includes single crystal X-ray diffraction (SXRD) and powder X-ray diffraction (PXRD). Main differences between the SXRD and the PXRD lie in a state and structure of a detection object. A detection object of the SXRD is usually a single crystal sample. A detection object of the PXRD is usually a powdered polycrystalline sample.

In the PXRD, copper (Cu) is typically used as the anode target material. When a high-energy electron beam bombards a copper target, inner-shell electrons are ionized, and when outer-shell electrons transition to fill vacancies in the inner-shell, a set of discontinuous characteristic X-ray spectra is emitted. Radiation emitted, when K-shell electrons are electrically excited and the resulting vacancies are filled by outer-shell electrons, is referred to as Cu-Kα radiation (i.e., Cu-Kα ray). Using Cu-Kα radiation for experiments can obtain an X-ray powder diffraction pattern (referred to as XRPD pattern), which typically exhibits a plurality of diffraction peaks.

In some embodiments, an X-ray powder diffraction pattern (Cu-Kα radiation) of the crystal form of acequinocyl represented by formula (I) has a characteristic diffraction peak at 3.3° 2θ±0.2° 2θ. It is understandable that the characteristic diffraction peak is a main characteristic diffraction peak of the crystal form of acequinocyl represented by formula (I) in embodiments of the present disclosure, which are determined based on a comprehensive evaluation of an angle, a d value, and an intensity of the peak. Therefore, by defining a position (at 3.3° 2θ±0.2° 2θ) of the characteristic diffraction peak, the crystal form of acequinocyl represented by formula (I) can be accurately identified, and the identification efficiency of the crystal form of acequinocyl represented by formula (I) can be improved.

Angle is a fundamental concept in mathematics for quantifying a size of an angle, which is measured in degrees (°) and reflects a rotational difference between two intersecting lines. “2θ” serves as an abscissa of the XRPD pattern and is a unified representation of a diffraction angle in XRPD technology. An origin of the “2θ” is directly related to Bragg's Law (i.e., 2d sin θ=nλ, where d is an interplanar spacing, θ is an incident angle, λ is an X-ray wavelength, and n is a diffraction order). As mentioned earlier, the “θ” is the incident angle, i.e., an angle between an incident direction of the X-ray and a crystal plane. However, a detector actually measures an angle between an incident ray and a diffracted ray after the X-ray irradiates a crystal. The angle is the 2θ (i.e., diffraction angle=2×incident angle). Therefore, the 2θ can be regarded as a standardized symbol for the diffraction angle and specifies that the “angle” described in embodiments of the present disclosure is a diffraction angle specific to X-ray diffraction experiments. Crystals of different crystal forms produce diffraction peaks at specific angles. The angles are related to the interplanar spacing of the crystals. Through Bragg's Law, the interplanar spacing of the crystals can be calculated, thereby enabling an inference of the crystal structures of the crystals. In addition, by analyzing intensities and shapes of diffraction peaks, information such as unit cell parameters, phase composition, crystal defects, and stress states of the crystals can also be determined.

It is understandable that in expressions similar to “3.3° 2θ±0.2° 2θ”, the 2θ can be regarded as a “unit” or a “corresponding coordinate system” for an angular value (e.g., 3.3°), emphasizing that such the angular value (e.g., 3.3°) is a value read from a diffraction angle scale (2θ). “±” indicates positive and negative deviation. For example, “±0.2° 2θ” indicates that an allowable deviation is plus or minus 0.2 degrees on the diffraction angle scale (2θ). Merely by way of example, the expression “a characteristic diffraction peak at 3.3° 2θ±0.2° 2θ” is interpreted as that a characteristic diffraction peak exists in the vicinity of diffraction angle of 3.3° 2θ, with an allowable measurement error of 0.2° 2θ.

In some embodiments, in addition to having the characteristic diffraction peak at 3.3° 2θ±0.2° 2θ, the X-ray powder diffraction pattern (Cu-Kα radiation) of the crystal form of acequinocyl represented by formula (I) further has a characteristic diffraction peak(s) at at least one of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ. For example, the X-ray powder diffraction pattern (Cu-Kα radiation) of the crystal form of acequinocyl represented by formula (I) has the characteristic diffraction peak at 3.3° 2θ±0.2° 2θ and a characteristic diffraction peak at 10.4° 2θ±0.2° 2θ. As another example, the X-ray powder diffraction pattern (Cu-Kα radiation) of the crystal form of acequinocyl represented by formula (I) has a characteristic diffraction peak at 3.3° 2θ±0.2° 2θ and a characteristic diffraction peak at 22.1° 2θ0.2° 2θ.

In some embodiments, in addition to having the characteristic diffraction peak at 3.3° 2θ±0.2° 2θ, the X-ray powder diffraction pattern (Cu-Kα radiation) of the crystal form of acequinocyl represented by formula (I) further has characteristic diffraction peaks at at least two of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ. For example, the X-ray powder diffraction pattern (Cu-Kα radiation) of the crystal form of acequinocyl represented by formula (I) has characteristic diffraction peaks at 3.3° 2θ±0.2° 2θ, 6.7° 2θ±0.2° 2θ, and 10.4° 2θ±0.2° 2θ. As another example, the X-ray powder diffraction pattern (Cu-Kα radiation) of the crystal form of acequinocyl represented by formula (I) has characteristic diffraction peaks at 3.3° 2θ±0.2° 2θ, 11.0° 2θ±0.2° 2θ, and 22.1° 2θ±0.2° 2θ.

In some embodiments, in addition to having the characteristic diffraction peak at 3.3° 2θ±0.2° 2θ, the X-ray powder diffraction pattern (Cu-Kα radiation) of the crystal form of acequinocyl represented by formula (I) further has characteristic diffraction peaks at at least three of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ. For example, the X-ray powder diffraction pattern (Cu-Kα radiation) of the crystal form of acequinocyl represented by formula (I) has characteristic diffraction peaks at 3.3° 2θ±0.2° 2θ, 6.7° 2θ±0.2° 2θ, 10.1° 2θ±0.2° 2θ, and 10.4° 2θ±0.2° 2θ. As another example, the X-ray powder diffraction pattern (Cu-Kα radiation) of the crystal form of acequinocyl represented by formula (I) has characteristic diffraction peaks at 3.3° 2θ±0.2° 2θ, 11.0° 2θ±0.2° 2θ, 22.1° 2θ±0.2° 2θ, and 24.5° 2θ±0.2° 2θ.

In some embodiments, in addition to having the characteristic diffraction peak at 3.3° 2θ±0.2° 2θ, the X-ray powder diffraction pattern (Cu-Kα radiation) of the crystal form of acequinocyl represented by formula (I) further has characteristic diffraction peaks at at least four of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ. For example, the X-ray powder diffraction pattern (Cu-Kα radiation) of the crystal form of acequinocyl represented by formula (I) has characteristic diffraction peaks at 3.3° 2θ±0.2° 2θ, 6.7° 2θ±0.2° 2θ, 10.1° 2θ±0.2° 2θ, 10.4° 2θ±0.2° 2θ, and 14.6° 2θ±0.2° 2θ. As another example, the X-ray powder diffraction pattern (Cu-Kα radiation) of the crystal form of acequinocyl represented by formula (I) has characteristic diffraction peaks at 3.3° 2θ±0.2° 2θ, 11.0° 2θ±0.2° 2θ, 13.5° 2θ±0.2° 2θ, 22.1° 2θ±0.2° 2θ, and 24.5° 2θ±0.2° 2θ.

In some embodiments, in addition to having the characteristic diffraction peak at 3.3° 2θ±0.2° 2θ, the X-ray powder diffraction pattern (Cu-Kα radiation) of the crystal form of acequinocyl represented by formula (I) has characteristic diffraction peaks at at least five of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ. For example, the X-ray powder diffraction pattern (Cu-Kα radiation) of the crystal form of acequinocyl represented by formula (I) has characteristic diffraction peaks at 3.3° 2θ±0.2° 2θ, 6.7° 2θ±0.2° 2θ, 10.1° 2θ±0.2° 2θ, 10.4° 2θ±0.2° 2θ, 11.0° 2θ±0.2° 2θ, and 14.6° 2θ±0.2° 2θ. As another example, the X-ray powder diffraction pattern (Cu-Kα radiation) of the crystal form of acequinocyl represented by formula (I) has characteristic diffraction peaks at 3.3° 2θ±0.2° 2θ, 11.0° 2θ±0.2° 2θ, 13.5° 2θ±0.2° 2θ, 14.6° 2θ±0.2° 2θ, 22.1° 2θ±0.2° 2θ, and 24.5° 2θ±0.2° 2θ.

In some embodiments, in addition to having the characteristic diffraction peak at 3.3° 2θ±0.2° 2θ, the X-ray powder diffraction pattern (Cu-Kα radiation) of the crystal form of acequinocyl represented by formula (I) has characteristic diffraction peaks at at least six of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ. For example, the X-ray powder diffraction pattern (Cu-Kα radiation) of the crystal form of acequinocyl represented by formula (I) has characteristic diffraction peaks at 3.3° 2θ±0.2° 2θ, 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, and 14.6° 2θ±0.2° 2θ. As another example, the X-ray powder diffraction pattern (Cu-Kα radiation) of the crystal form of acequinocyl represented by formula (I) has characteristic diffraction peaks at 3.3° 2θ±0.2° 2θ, 6.7° 2θ±0.2° 2θ, 11.0° 2θ±0.2° 2θ, 13.5° 2θ±0.2° 2θ, 14.6° 2θ±0.2° 2θ, 22.1° 2θ±0.2° 2θ, and 24.5° 2θ±0.2° 2θ.

In some embodiments, in addition to having the characteristic diffraction peak at 3.3° 2θ±0.2° 2θ, the X-ray powder diffraction pattern (Cu-Kα radiation) of the crystal form of acequinocyl represented by formula (I) has characteristic diffraction peaks at at least seven of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ. For example, the X-ray powder diffraction pattern (Cu-Kα radiation) of the crystal form of acequinocyl represented by formula (I) has characteristic diffraction peaks at 3.3° 2θ±0.2° 2θ, 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, and 17.1° 2θ±0.2° 2θ. As another example, the X-ray powder diffraction pattern (Cu-Kα radiation) of the crystal form of acequinocyl represented by formula (I) has characteristic diffraction peaks at 3.3° 2θ±0.2° 2θ, 6.7° 2θ±0.2° 2θ, 10.4° 2θ±0.2° 2θ, 11.0° 2θ±0.2° 2θ, 13.5° 2θ±0.2° 2θ, 17.1° 2θ±0.2° 2θ, 22.1° 2θ±0.2° 2θ, and 24.5° 2θ±0.2° 2θ.

In some embodiments, in addition to having the characteristic diffraction peak at 3.3° 2θ±0.2° 2θ, the X-ray powder diffraction pattern (Cu-Kα radiation) of the crystal form of acequinocyl represented by formula (I) has characteristic diffraction peaks at at least eight of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ. For example, the X-ray powder diffraction pattern (Cu-Kα radiation) of the crystal form of acequinocyl represented by formula (I) has characteristic diffraction peaks at 3.3° 2θ±0.2° 2θ, 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, and 24.5° 2θ±0.2° 2θ. As another example, the X-ray powder diffraction pattern (Cu-Kα radiation) of the crystal form of acequinocyl represented by formula (I) has characteristic diffraction peaks at 3.3° 2θ±0.2° 2θ, 6.7° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, and 24.5° 2θ±0.2° 2θ.

In some embodiments, in addition to having the characteristic diffraction peak at 3.3° 2θ±0.2° 2θ, the X-ray powder diffraction pattern (Cu-Kα radiation) of the crystal form of acequinocyl represented by formula (I) has characteristic diffraction peaks at 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, and 24.5° 2θ±0.2° 2θ. It can be understood that, under certain circumstances, the crystal form of acequinocyl represented by formula (I) described in the present disclosure may include all of the above characteristic diffraction peaks.

In some embodiments, the X-ray powder diffraction pattern (Cu-Kα radiation) of the crystal form of acequinocyl represented by formula (I) has characteristic diffraction peaks at 3.3465° 2θ±0.2° 2θ, 6.7245° 2θ±0.2° 2θ, 10.1118° 2θ±0.2° 2θ, 10.4289° 2θ±0.2° 2θ, 10.9607° 2θ±0.2° 2θ, 13.5235° 2θ±0.2° 2θ, 14.5662° 2θ±0.2° 2θ, 17.1189° 2θ±0.2° 2θ, 22.0684° 2θ±0.2° 2θ, and 24.5351° 2θ±0.2° 2θ.

In some embodiments, the X-ray powder diffraction pattern (Cu-Kα radiation) of the crystal form of acequinocyl represented by formula (I) has characteristic diffraction peaks at 3.3513° 2θ±0.2° 2θ, 6.7269° 2θ±0.2° 2θ, 10.1144° 2θ±0.2° 2θ, 10.4262° 2θ±0.2° 2θ, 10.9626° 2θ±0.2° 2θ, 13.5256° 2θ±0.2° 2θ, 14.5699° 2θ±0.2° 2θ, 17.1275° 2θ±0.2° 2θ, 22.0641° 2θ±0.2° 2θ, and 24.5299° 2θ±0.2° 2θ.

In some embodiments, the X-ray powder diffraction pattern (Cu-Kα radiation) of the crystal form of acequinocyl represented by formula (I) has characteristic diffraction peaks at 3.3874° 2θ±0.2° 2θ, 6.7684° 2θ±0.2° 2θ, 10.1553° 2θ±0.2° 2θ, 10.4614° 2θ±0.2° 2θ, 10.9971° 2θ±0.2° 2θ, 13.5633° 2θ±0.2° 2θ, 14.6231° 2θ±0.2° 2θ, 17.1483° 2θ±0.2° 2θ, 22.0848° 2θ±0.2° 2θ, and 24.5728° 2θ±0.2° 2θ.

In some embodiments, the X-ray powder diffraction pattern (Cu-Kα radiation) of the crystal form of acequinocyl represented by formula (I) has characteristic diffraction peaks at 3.3794° 2θ±0.2° 2θ, 6.7564° 2θ±0.2° 2θ, 10.1493° 2θ±0.2° 2θ, 10.4483° 2θ±0.2° 2θ, 10.9867° 2θ±0.2° 2θ, 13.5568° 2θ±0.2° 2θ, 14.5973° 2θ±0.2° 2θ, 17.1544° 2θ±0.2° 2θ, 22.0885° 2θ±0.2° 2θ, and 24.5680° 2θ±0.2° 2θ.

By defining positions of the characteristic diffraction peaks, information related to a crystal structure of the crystal form of acequinocyl represented by formula (I) can be accurately determined based on the XRPD. More details regarding the X-ray powder diffraction pattern of the crystal form of acequinocyl represented by formula (I) may be found in the descriptions related to Embodiments 1-4, and are not repeated herein. Furthermore, by further defining positions of other characteristic diffraction peaks of the crystal form of acequinocyl represented by formula (I) on the basis of specifying the main characteristic diffraction peak (3.3° 2θ±0.2° 2θ), the identification process of the crystal form of acequinocyl represented by formula (I) in the embodiments of the present disclosure can be made more accurate.

In XRPD technology, a relative intensity refers to a parameter in which an intensity of a strongest diffraction peak in the X-ray powder diffraction pattern is normalized to 100%, and intensities of other diffraction peaks are expressed as percentages relative to the strongest peak. It is understandable that the intensity of the diffraction peak reflects the diffraction ability of a crystal to the X-rays, and is related to the orderliness of an arrangement of corresponding crystal planes in the crystal and a count of crystal planes (affected by crystal's preferred orientation). The higher the orderliness of the arrangement of the corresponding crystal planes and the greater the count of crystal planes in this direction, the higher the intensity of the diffraction peak.

In some embodiments, a relative intensity of the characteristic diffraction peak at 3.3° 2θ±0.2° 2θ is defined as 100%, and relative intensities of characteristic diffraction peaks at 10.1° 2θ±0.2° 2θ and 10.4° 2θ±0.2° 2θ are greater than 5%. In some embodiments, the relative intensity of the characteristic diffraction peak at 3.3° 2θ±0.2° 2θ is defined as 100%, a relative intensity of a characteristic diffraction peak at 10.1° 2θ±0.2° 2θ is greater than 6%, e.g., greater than 7%, greater than 8%, greater than 10%, greater than 15%, or greater than 20%. In some embodiments, the relative intensity of the characteristic diffraction peak at 3.3° 2θ±0.2° 2θ is defined as 100%, a relative intensity of a characteristic diffraction peak at 10.4° 2θ±0.2° 2θ is greater than 5.5%, e.g., greater than 9%, greater than 10%, greater than 15%, greater than 20%, greater than 25%, greater than 30%, greater than 35%, greater than 40%, greater than 45%, or greater than 50%.

In some embodiments, the relative intensity of the characteristic diffraction peak at 3.3° 2θ±0.2° 2θ is defined as 100%, relative intensities of characteristic diffraction peaks at 11.0° 2θ±0.2° 2θ, 13.5° 2θ±0.2° 2θ, and 22.1° 2θ±0.2° 2θ are greater than 3%. In some embodiments, the relative intensity of the characteristic diffraction peak at 3.3° 2θ±0.2° 2θ is defined as 100%, a relative intensity of a characteristic diffraction peak at 11.0° 2θ±0.2° 2θ is greater than 3.5%, e.g., greater than 5%, greater than 6%, greater than 10%, greater than 15%, greater than 20%, greater than 25%, greater than 30%, or greater than 35%. In some embodiments, the relative intensity of the characteristic diffraction peak at 3.3° 2θ±0.2° 2θ is defined as 100%, a relative intensity of a characteristic diffraction peak at 13.5° 2θ±0.2° 2θ is greater than 4%, e.g., greater than 5%, greater than 6%, greater than 6.3%, greater than 10%, greater than 15%, greater than 20%, or greater than 25%. In some embodiments, the relative intensity of the characteristic diffraction peak at 3.3° 2θ±0.2° 2θ is defined as 100%, a relative intensity of a characteristic diffraction peak at 22.1° 2θ±0.2° 2θ is greater than 4%, e.g., greater than 5%, greater than 10%, greater than 15%, greater than 20%, greater than 25%, greater than 30%, greater than 35%, greater than 40%, or greater than 45%.

In some embodiments, the relative intensity of the characteristic diffraction peak at 3.3° 2θ±0.2° 2θ is defined as 100%, and relative intensities of characteristic diffraction peaks at 6.7° 2θ±0.2° 2θ, 14.6° 2θ±0.2° 2θ, 17.1° 2θ±0.2° 2θ, and 24.5° 2θ±0.2° 2θ are greater than 1%. In some embodiments, the relative intensity of the characteristic diffraction peak at 3.3° 2θ±0.2° 2θ is defined as 100%, a relative intensity of a characteristic diffraction peak at 6.7° 2θ±0.2° 2θ is greater than 1.4%, e.g., greater than 1.7%, greater than 2%, or greater than 2.5%. In some embodiments, the relative intensity of the characteristic diffraction peak at 3.3° 2θ±0.2° 2θ is defined as 100%, a relative intensity of a characteristic diffraction peak at 14.6° 2θ±0.2° 2θ is greater than 1.1%, e.g., greater than 1.5%, greater than 1.7%, greater than 2%, greater than 4%, greater than 6%, greater than 8%, greater than 10%, or greater than 12%. In some embodiments, the relative intensity of the characteristic diffraction peak at 3.3° 2θ±0.2° 2θ is defined as 100%, a relative intensity of a characteristic diffraction peak at 17.1° 2θ±0.2° 2θ is greater than 1.9%, e.g., greater than 2%, greater than 2.8%, greater than 3%, greater than 4%, greater than 6%, greater than 8%, greater than 10%, greater than 12%, greater than 14%, greater than 16%, or greater than 20%. In some embodiments, the relative intensity of the characteristic diffraction peak at 3.3° 2θ±0.2° 2θ is defined as 100%, a relative intensity of a characteristic diffraction peak at 24.5° 2θ±0.2° 2θ is greater than 1.5%, e.g., greater than 2%, greater than 2.5%, greater than 2.7%, greater than 3%, greater than 5%, greater than 10%, greater than 15%, greater than 20%, greater than 25%, greater than 30%, greater than 35%, greater than 40%, greater than 45%, or greater than 50%.

It is understandable that the relative intensity of the characteristic diffraction peak at 3.3° 2θ±0.2° 2θ is taken as a reference (100%), because this peak is a characteristic strong peak of the target crystal form (i.e., the crystal form of acequinocyl represented by formula (I)). It features a high diffraction ability and a relatively stable signal, and using the characteristic diffraction peak as a benchmark allows for a more accurate quantification of the relative intensities of other characteristic diffraction peaks. On the basis, by further defining the relative intensities of the characteristic diffraction peaks at 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, and/or 24.5° 2θ±0.2° 2θ enables the clarification of diffraction angle positions while introducing intensity quantification indicators, and establishes a dual precise identification system of “diffraction angle position+relative intensity”. This addresses the difficulty in distinguishing crystal forms when relying solely on diffraction angle positions in certain cases, and thus ensures the purity and specificity of the crystal form of acequinocyl represented by formula (I).

In some embodiments, a relative intensity of a characteristic diffraction peak at 3.3465° 2θ±0.2° 2θ is defined as 100%, and relative intensities of characteristic diffraction peaks at 6.7245° 2θ±0.2° 2θ, 10.1118° 2θ±0.2° 2θ, 10.4289° 2θ±0.2° 2θ, 10.9607° 2θ±0.2° 2θ, 13.5235° 2θ±0.2° 2θ, 14.5662° 2θ±0.2° 2θ, 17.1189° 2θ±0.2° 2θ, 22.0684° 2θ±0.2° 2θ, and 24.5351° 2θ±0.2° 2θ are 1.88%, 8%, 10.52%, 6.65%, 6.47%, 1.72%, 2.86%, 5.9%, and 2.56%, respectively.

In some embodiments, a relative intensity of a characteristic diffraction peak at 3.3513° 2θ±0.2° 2θ is defined as 100%, and relative intensities of characteristic diffraction peaks at 6.7269° 2θ±0.2° 2θ, 10.1144° 2θ±0.2° 2θ, 10.4262° 2θ±0.2° 2θ, 10.9626° 2θ±0.2° 2θ, 13.5256° 2θ±0.2° 2θ, 14.5699° 2θ±0.2° 2θ, 17.1275° 2θ±0.2° 2θ, 22.0641° 2θ±0.2° 2θ, and 24.5299° 2θ±0.2° 2θ are 1.77%, 8.54%, 16.7%, 11.03%, 5.65%, 2.09%, 3.36%, 10.19%, and 3.73%, respectively.

In some embodiments, a relative intensity of a characteristic diffraction peak at 3.3874° 2θ±0.2° 2θ is defined as 100%, and relative intensities of characteristic diffraction peaks at 6.7684° 2θ±0.2° 2θ, 10.1553° 2θ±0.2° 2θ, 10.4614° 2θ±0.2° 2θ, 10.9971° 2θ±0.2° 2θ, 13.5633° 2θ±0.2° 2θ, 14.6231° 2θ±0.2° 2θ, 17.1483° 2θ±0.2° 2θ, 22.0848° 2θ±0.2° 2θ, and 24.5728° 2θ±0.2° 2θ are 2.04%, 7.03%, 9.41%, 5.88%, 6.25%, 1.58%, 2.13%, 4.65%, and 1.55%, respectively.

In some embodiments, a relative intensity of a characteristic diffraction peak at 3.3794° 2θ±0.2° 2θ is defined as 100%, and relative intensities of characteristic diffraction peaks at 6.7564° 2θ±0.2° 2θ, 10.1493° 2θ±0.2° 2θ, 10.4483° 2θ±0.2° 2θ, 10.9867° 2θ±0.2° 2θ, 13.5568° 2θ±0.2° 2θ, 14.5973° 2θ±0.2° 2θ, 17.1544° 2θ±0.2° 2θ, 22.0885° 2θ±0.2° 2θ, and 24.5680° 2θ±0.2° 2θ are 1.5%, 6.18%, 5.62%, 3.81%, 4.67%, 1.2%, 1.99%, 3.49%, and 2.74%, respectively.

In some embodiments of the present disclosure, providing the crystal form of the acequinocyl represented by formula (I):

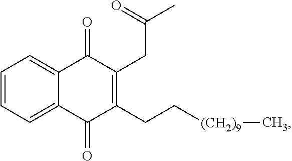

and the X-ray powder diffraction pattern (Cu-Kα radiation) of the crystal form has the characteristic diffraction peak at 3.3° 2θ±0.2° 2θ and the characteristic diffraction peak(s) at at least one of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ, thus a new crystalline form of acequinocyl represented by formula (I) is proposed. Compared with existing acequinocyl crystals, the crystal form of acequinocyl represented by formula (I) exhibits a higher melting point and a higher initial melting temperature, as well as a narrower melting range. These properties prevent the occurrence of creaming and similar phenomena during the preparation of aqueous suspension concentrates using the crystal form of acequinocyl represented by formula (I), thereby yielding the acequinocyl suspension concentrates prepared therefrom with higher quality, superior performance, and an extended storage life. Meanwhile, they also improve the applicability of acequinocyl suspension concentrate products to a certain extent.

Thermal analysis refers to a method that utilizes physical changes (e.g., melting, evaporation, etc.) or chemical changes (e.g., thermal decomposition, oxidation, etc.) occurring in a substance during heating or cooling, and determines a crystal form by analyzing measured thermal analysis thermograms (e.g., differential scanning calorimetry thermograms, thermogravimetric thermograms, etc.). Thermal analysis may include differential scanning calorimetry (DSC), thermogravimetric analysis (TGA), or the like.

The DSC is a method for measuring a relationship between a thermal power difference between a sample and a reference and a temperature. When the sample undergoes a thermal reaction (e.g., an endothermic or exothermic reaction), a DSC system records a heat change and generates a differential scanning calorimetry thermogram (referred to as a DSC thermogram). The DSC thermogram takes a heat flow rate (defined as heat absorbed or released by a unit mass of a sample per unit time, in milliwatts per milligram, i.e., mW/mg) as a vertical coordinate, and takes a temperature T as a horizontal coordinate. By further analyzing the DSC thermogram, various thermal properties (e.g., phase transition, melting point, crystallization, chemical reaction, and glass transition) of the sample during a temperature change process can be determined.

In some embodiments, a differential scanning calorimetry thermogram of the crystal form of the acequinocyl represented by formula (I), obtained at a heating rate of 5° C./min, has at least one characteristic thermal event in a range of 55° C. to 60° C. The characteristic thermal event refers to an event that occurs and reflects an endothermic or exothermic phenomenon associated with a change in the thermal properties of the substance (i.e., the crystal form of the acequinocyl represented by formula (I)) at a specific temperature. For example, the characteristic thermal event includes an endothermic event and/or an exothermic event. The endothermic event appears as an upward peak on the differential scanning calorimetry thermogram, indicating that the sample absorbs heat over a certain temperature range, and this is commonly observed in processes such as melting, decomposition, desorption, and the like. The exothermic event appears as a downward peak on the differential scanning calorimetry thermogram, indicating that the sample releases heat over a certain temperature range, and this is commonly observed in processes such as crystallization, oxidation, solidification, and the like.

In some embodiments, the differential scanning calorimetry thermogram of the crystal form of the acequinocyl represented by formula (I), obtained at the heating rate of 5° C./min, has at least one characteristic thermal event in a range of 55.5° C. to 59.5° C. In some embodiments, the differential scanning calorimetry thermogram of the crystal form of the acequinocyl represented by formula (I), obtained at the heating rate of 5° C./min, has at least one characteristic thermal event in a range of 56° C. to 59° C. In some embodiments, the differential scanning calorimetry thermogram of the crystal form of the acequinocyl represented by formula (I), obtained at the heating rate of 5° C./min, has at least one characteristic thermal event in a range of 56.5° C. to 58.5° C. In some embodiments, the differential scanning calorimetry thermogram of the crystal form of the acequinocyl represented by formula (I), obtained at the heating rate of 5° C./min, has at least one characteristic thermal event in a range of 57.0° C. to 58.5° C.

In some embodiments, the differential scanning calorimetry thermogram of the crystal form of the acequinocyl represented by formula (I), obtained at the heating rate of 5° C./min, has at least one characteristic thermal event in a range of 56° C. to 58° C. In some embodiments, the differential scanning calorimetry thermogram of the crystal form of the acequinocyl represented by formula (I), obtained at the heating rate of 5° C./min, has at least one characteristic thermal event in a range of 56.5° C. to 58° C. In some embodiments, the differential scanning calorimetry thermogram of the crystal form of the acequinocyl represented by formula (I), obtained at the heating rate of 5° C./min, has at least one characteristic thermal event in a range of 56.96° C. to 57.55° C.

In some embodiments, the differential scanning calorimetry thermogram of the crystal form of the acequinocyl represented by formula (I), obtained at the heating rate of 5° C./min, has at least one characteristic thermal event in a range of 57° C. to 59° C. In some embodiments, the differential scanning calorimetry thermogram of the crystal form of the acequinocyl represented by formula (I), obtained at the heating rate of 5° C./min, has at least one characteristic thermal event in a range of 57.5° C. to 59° C. In some embodiments, the differential scanning calorimetry thermogram of the crystal form of the acequinocyl represented by formula (I), obtained at the heating rate of 5° C./min, has at least one characteristic thermal event in a range of 57.84° C. to 58.51° C.

In some embodiments, the differential scanning calorimetry thermogram of the crystal form of the acequinocyl represented by formula (I), obtained at the heating rate of 5° C./min, has at least one characteristic thermal event in a range of 57° C. to 58° C. In some embodiments, the differential scanning calorimetry thermogram of the crystal form of the acequinocyl represented by formula (I), obtained at the heating rate of 5° C./min, has at least one characteristic thermal event in a range of 57.2° C. to 57.8° C. In some embodiments, the differential scanning calorimetry thermogram of the crystal form of the acequinocyl represented by formula (I), obtained at the heating rate of 5° C./min, has at least one characteristic thermal event in a range of 57.4° C. to 57.6° C. In some embodiments, the differential scanning calorimetry thermogram of the crystal form of the acequinocyl represented by formula (I), obtained at the heating rate of 5° C./min, has at least one characteristic thermal event in a range of 57.03° C. to 57.81° C. In some embodiments, the differential scanning calorimetry thermogram of the crystal form of the acequinocyl represented by formula (I), obtained at the heating rate of 5° C./min, has at least one characteristic thermal event in a range of 57.32° C. to 57.58° C.

In some embodiments, the differential scanning calorimetry thermogram of the crystal form of the acequinocyl represented by formula (I), obtained at the heating rate of 5° C./min, has at least one characteristic thermal event in a range of 58° C. to 59° C. In some embodiments, the differential scanning calorimetry thermogram of the crystal form of the acequinocyl represented by formula (I), obtained at the heating rate of 5° C./min, has at least one characteristic thermal event in a range of 58.2° C. to 58.8° C. In some embodiments, the differential scanning calorimetry thermogram of the crystal form of the acequinocyl represented by formula (I), obtained at the heating rate of 5° C./min, has at least one characteristic thermal event in a range of 58.4° C. to 58.6° C.

In some embodiments, the differential scanning calorimetry thermogram of the crystal form of the acequinocyl represented by formula (I), obtained at the heating rate of 5° C./min, has at least one characteristic thermal event in a range of 57.03° C. to 57.81° C. In some embodiments, the differential scanning calorimetry thermogram of the crystal form of the acequinocyl represented by formula (I), obtained at the heating rate of 5° C./min, has at least one characteristic thermal event in a range of 56.96° C. to 57.55° C. In some embodiments, the differential scanning calorimetry thermogram of the crystal form of the acequinocyl represented by formula (I), obtained at the heating rate of 5° C./min, has at least one characteristic thermal event in a range of 57.84° C. to 58.51° C. In some embodiments, the differential scanning calorimetry thermogram of the crystal form of the acequinocyl represented by formula (I), obtained at the heating rate of 5° C./min, has at least one characteristic thermal event in a range of 57.32° C. to 57.58° C.

In some embodiments, in addition to having at least one characteristic thermal event in the range of 55° C. to 60° C., the differential scanning calorimetry thermogram of the crystal form of the acequinocyl represented by formula (I), obtained at the heating rate of 5° C./min, has characteristic thermal events in other temperature ranges. For example, the differential scanning calorimetry thermogram of the crystal form of the acequinocyl represented by formula (I) has one characteristic thermal event each in a range of 54.14° C. to 54.89° C. and in a range of 57.32° C. to 57.58° C.

In some embodiments of the present disclosure, by limiting the temperature range of the characteristic thermal event in the differential scanning calorimetry thermogram of the crystal form of the acequinocyl represented by formula (I), thermodynamic-related information of the crystal form of the acequinocyl represented by formula (I) can be accurately determined based on the DSC. The thermodynamic-related information, together with crystal structure-related information obtained by the aforementioned XRPD method, can be used to jointly determine the crystal form of the acequinocyl represented by formula (I) described in the embodiments of the present disclosure. More details regarding the differential scanning calorimetry thermogram may be found in the description related to Embodiments 1-4, and are not repeated herein.

In some embodiments, an aqueous suspension concentrate prepared from the crystal form of the acequinocyl represented by formula (I) has a particle size increase of less than a threshold after thermal storage at a certain temperature for a count of days and subsequent storage at room temperature for a count of days. The certain temperature may be greater than 25° C., e.g., greater than 50° C., greater than 53° C., or equal to 54° C. The count of days may be greater than 10 days, e.g., greater than 15 days, or greater than 30 days. The threshold of the particle size increase may be 300%, 250%, or 200%. In some embodiments, the aqueous suspension concentrate prepared from the crystal form of the acequinocyl represented by formula (I) has a particle size increase of less than 300% after thermal storage at a temperature greater than 50° C. for over 10 days and subsequent storage at the room temperature for not less than 30 days. For example, the aqueous suspension concentrate prepared from the crystal form of the acequinocyl represented by formula (I) has a particle size increase of less than 300% after thermal storage at 54° C. for 14 days and subsequent storage at the room temperature for 1 month. As another example, the aqueous suspension concentrate prepared from the crystal form of the acequinocyl represented by formula (I) has a particle size increase of less than 250% after thermal storage at 54° C. for 14 days and subsequent storage at the room temperature for 1 month. As another example, the aqueous suspension concentrate prepared from the crystal form of the acequinocyl represented by formula (I) has a particle size increase of less than 200% after thermal storage at 54° C. for 14 days and subsequent storage at the room temperature for 1 month. As another example, the aqueous suspension concentrate prepared from the crystal form of the acequinocyl represented by formula (I) has a particle size increase of less than 150% after thermal storage at 54° C. for 14 days and subsequent storage at the room temperature for 1 month. As another example, the aqueous suspension concentrate prepared from the crystal form of the acequinocyl represented by formula (I) has a particle size increase of less than 100% after thermal storage at 54° C. for 14 days and subsequent storage at the room temperature for 1 month. As another example, the aqueous suspension concentrate prepared from the crystal form of the acequinocyl represented by formula (I) has a particle size increase of less than 50% after thermal storage at 54° C. for 14 days and subsequent storage at the room temperature for 1 month.

In some embodiments, the aqueous suspension concentrate prepared from the crystal form of the acequinocyl represented by formula (I) has a particle size increase of not more than 131.2% after thermal storage at 54° C. for 14 days and subsequent storage at the room temperature for 2 hours. In some embodiments, the aqueous suspension concentrate prepared from the crystal form of the acequinocyl represented by formula (I) has a particle size increase of not more than 65.8% after thermal storage at 54° C. for 14 days and subsequent storage at the room temperature for 2 hours. In some embodiments, the aqueous suspension concentrate prepared from the crystal form of the acequinocyl represented by formula (I) has a particle size increase of not more than 60.8% after thermal storage at 54° C. for 14 days and subsequent storage at the room temperature for 2 hours. In some embodiments, the aqueous suspension concentrate prepared from the crystal form of the acequinocyl represented by formula (I) has a particle size increase of not more than 48.9% after thermal storage at 54° C. for 14 days and subsequent storage at the room temperature for 2 hours.

In some embodiments, the aqueous suspension concentrate prepared from the crystal form of the acequinocyl represented by formula (I) has a particle size increase of not more than 196.4% after thermal storage at 54° C. for 14 days and subsequent storage at the room temperature for 14 days. In some embodiments, the aqueous suspension concentrate prepared from the crystal form of the acequinocyl represented by formula (I) has a particle size increase of not more than 102.8% after thermal storage at 54° C. for 14 days and subsequent storage at the room temperature for 14 days. In some embodiments, the aqueous suspension concentrate prepared from the crystal form of the acequinocyl represented by formula (I) has a particle size increase of not more than 91.3% after thermal storage at 54° C. for 14 days and subsequent storage at the room temperature for 14 days. In some embodiments, the aqueous suspension concentrate prepared from the crystal form of the acequinocyl represented by formula (I) has a particle size increase of not more than 73.7% after thermal storage at 54° C. for 14 days and subsequent storage at the room temperature for 14 days.

In some embodiments, the aqueous suspension concentrate prepared from the crystal form of the acequinocyl represented by formula (I) has a particle size increase of not more than 264.0% after thermal storage at 54° C. for 14 days and subsequent storage at the room temperature for 30 days. In some embodiments, the aqueous suspension concentrate prepared from the crystal form of the acequinocyl represented by formula (I) has a particle size increase of not more than 188.2% after thermal storage at 54° C. for 14 days and subsequent storage at the room temperature for 30 days. In some embodiments, the aqueous suspension concentrate prepared from the crystal form of the acequinocyl represented by formula (I) has a particle size increase of not more than 167.0% after thermal storage at 54° C. for 14 days and subsequent storage at the room temperature for 30 days. In some embodiments, the aqueous suspension concentrate prepared from the crystal form of the acequinocyl represented by formula (I) has a particle size increase of not more than 76.2% after thermal storage at 54° C. for 14 days and subsequent storage at the room temperature for 30 days.

The thermal storage refers to storing a sealed formulation sample in a temperature-controlled device (e.g., a constant temperature incubator, an oven, etc.). It is understandable that the principle of thermal storage is to simulate changes that may occur during long-term storage at room temperature by conducting short-term storage under elevated temperature conditions. According to the Arrhenius equation for chemical reactions, a reaction rate increases by approximately 2 to 4 times for every 10° C. rise in temperature. Raising the storage temperature significantly from the room temperature (e.g., 25° C.) to 50° C. can greatly accelerate potential unstable processes in the formulation (e.g., active ingredient degradation, particle aggregation/growth, phase separation, or creaming, etc.), thus enabling the prediction of the formulation's stability at room temperature over months or even years within a few weeks. More details regarding the thermal storage stability test may be found in the following related description.

Particle size refers to a size of solid active ingredient particles (i.e., the crystal form of the acequinocyl represented by formula (I) in the suspension concentrate. Particle size increase refers to a parameter indicating a degree of particle size growth during storage. Specifically, the particle size increase refers to a percentage of a difference between an average particle size after storage and an average particle size before the storage (i.e., an initial average particle size) relative to the initial average particle size, that is, particle size increase=(average particle size after storage−initial average particle size)/initial average particle size×100%. It is understandable that particle size increase may be caused by two mechanisms. The first mechanism is the Ostwald ripening mechanism. During storage, small particles in the solid phase dissolve due to their higher solubility, and solute recrystallizes on surfaces of large particles, causing the large particles to grow larger and the small particles to disappear, which results in an overall increase in the average particle size. The second mechanism is the crystal aggregation mechanism. Solid particles in the suspension concentrate need to be stably dispersed in a solvent phase. If the interparticle attractive forces are greater than dispersion forces, agglomeration may occur (the small particles aggregate to form the large particles), leading to an increase in the particle size.

It is understandable that a low particle size increase (e.g., the particle size increase is less than 300%) means that a formulation system can maintain uniformity and dispersion of a particle state even under elevated temperature conditions, which serves as a direct guarantee against problems such as creaming, sedimentation, and nozzle blockage. In some embodiments of the present disclosure, controlling the particle size increase to be less than 300% in the thermal storage stability test can ensure the long-term storage stability of the formulation of the crystal form of the acequinocyl represented by formula (I), and extend the product shelf life.

Embodiments of the present disclosure provide a method for preparing the crystal form of the acequinocyl represented by formula (I) described in the embodiments of the present disclosure. In some embodiments, the method may include a first method (also referred to as a method a), a second method (also referred to as a method b), or a third method (also referred to as a method c).

In some embodiments, the first method, the second method, and the third method may each be performed in a reaction vessel of a certain volume. For example, the first method, the second method, and the third method may each be performed in a 10 L reaction kettle.

In some embodiments, the first method includes dissolving the acequinocyl represented by formula (I) in a crystallization solvent, heating to 55° C. to 70° C., cooling to crystallize, and filtering to obtain the crystal form of the acequinocyl represented by formula (I).

Preferably, the first method includes heating to 56° C. to 69° C. Preferably, the first method includes heating to 57° C. to 68° C. Preferably, the first method includes heating to 58° C. to 67° C. Preferably, the first method includes heating to 59° C. to 66° C. Preferably, the first method includes heating to 60° C. to 65° C. Preferably, the first method includes heating to 61° C. to 64° C. Preferably, the first method includes heating to 62° C. to 63° C. Preferably, the first method includes heating to 60° C.

The crystallization solvent refers to a liquid used in a crystallization process that can assist a solute in precipitating to form a crystal. In some embodiments, the crystallization solvent includes at least one of water, alcohol, ether, or hydrocarbon. In some embodiments, the alcohol includes at least one of methanol, ethanol, or isopropanol. In some embodiments, the ether may include 2-methyltetrahydrofuran. In some embodiments, the hydrocarbon includes at least one of n-hexane, cyclohexane, n-pentane, or n-heptane. In some embodiments, preferably, the crystallization solvent includes at least one of methanol, ethanol, or 2-methyltetrahydrofuran. It is understandable that selecting a suitable crystallization solvent is a key factor in ensuring crystal quality and improving crystallization efficiency. By limiting preferred crystallization solvents, the crystallization process of the crystal form of the acequinocyl represented by formula (I) described in the embodiments of the present disclosure can be carried out smoothly, and high-quality crystals can be obtained as far as possible.

In some embodiments, in the first method, a weight ratio of the crystallization solvent to the acequinocyl represented by formula (I) may be (0.5-10):1. It is understandable that if the weight ratio of the crystallization solvent to the acequinocyl represented by formula (I) is too high (i.e., the crystallization solvent is excessive), a hot saturated solution may fail to form, causing acequinocyl represented by formula (I) to fail to precipitate in crystalline form or not precipitating at all upon cooling, leading to a decrease in the amount of crystallized product and a lower yield. If the weight ratio of the crystallization solvent to the acequinocyl represented by formula (I) is too low (i.e., the crystallization solvent is insufficient), a portion of acequinocyl represented by formula (I) may fail to dissolve completely upon heating, thus remaining in the finished crystals and compromising the crystal quality. Setting the weight ratio of the crystallization solvent to the acequinocyl represented by formula (I) at (0.5-10):1 ensures an appropriate solute-solvent ratio and facilitates the subsequent crystallization process. Preferably, in the first method, the weight ratio of the crystallization solvent to the acequinocyl represented by formula (I) is (0.7-8):1. Preferably, in the first method, the weight ratio of the crystallization solvent to the acequinocyl represented by formula (I) is (0.9-6):1. Preferably, in the first method, the weight ratio of the crystallization solvent to the acequinocyl represented by formula (I) is (1-5):1. Preferably, in the first method, the weight ratio of the crystallization solvent to the acequinocyl represented by formula (I) is (2-4):1. Preferably, in the first method, the weight ratio of the crystallization solvent to the acequinocyl represented by formula (I) is 3:1. By setting a preferred weight ratio, a favorable solute-solvent ratio can be determined, enabling the crystal quality to be as excellent as possible and the crystallization yield to be as high as possible.

The crystallization temperature refers to a temperature at which crystallization occurs (i.e., a temperature after the aforementioned cooling). In some embodiments, the crystallization temperature of the acequinocyl represented by formula (I) may be 0° C. to 70° C. It is understandable that temperature changes directly affect crystallization processes and their outcomes. An excessively high or low crystallization temperature may result in crystallization failure. If the crystallization temperature is excessively high, the substance may melt, lose its crystal structure, and fail to form stable crystals. If the crystallization temperature is excessively low, the molecular motion of the substance will slow down, and the crystallization rate will decrease or even stop. By setting the crystallization temperature of the acequinocyl represented by formula (I) within the range of 0° C. to 70° C., normal crystal precipitation can be ensured, and adverse consequences such as crystal defects and a low crystallization yield caused by an inappropriate crystallization temperature can be avoided. Preferably, the crystallization temperature of the acequinocyl represented by formula (I) is 1° C. to 60° C. Preferably, the crystallization temperature of the acequinocyl represented by formula (I) is 2° C. to 50° C. Preferably, the crystallization temperature of the acequinocyl represented by formula (I) is 3° C. to 40° C. Preferably, the crystallization temperature of the acequinocyl represented by formula (I) is 4° C. to 30° C. Preferably, the crystallization temperature of the acequinocyl represented by formula (I) is 5° C. to 20° C. Preferably, the crystallization temperature of the acequinocyl represented by formula (I) is 6° C. to 10° C. Preferably, the crystallization temperature of the acequinocyl represented by formula (I) is 5° C. to 10° C. Preferably, the crystallization temperature of the acequinocyl represented by formula (I) is 7° C. to 9° C. Preferably, the crystallization temperature of the acequinocyl represented by formula (I) is 8° C. to 9° C. By setting a preferred crystallization temperature, a crystal precipitation rate can be appropriate, and the crystal quality can be optimized as much as possible.

More details regarding the first method may be found in the descriptions related to Embodiments 1, 2, and 4.

In some embodiments, the second method includes heating the acequinocyl represented by formula (I) to 55° C. to 70° C. to melt, cooling and slicing to obtain the crystal form of the acequinocyl represented by formula (I).

Preferably, the second method includes heating to 56° C. to 69° C. Preferably, the second method includes heating to 57° C. to 68° C. Preferably, the second method includes heating to 58° C. to 67° C. Preferably, the second method includes heating to 59° C. to 66° C. Preferably, the second method includes heating to 60° C. to 65° C. Preferably, the second method includes heating to 61° C. to 64° C. Preferably, the second method includes heating to 52° C. to 63° C. Preferably, the second method includes heating to 60° C. to 70° C. Preferably, the second method includes heating to 61° C. to 69° C. Preferably, the second method includes heating to 62° C. to 68° C. Preferably, the second method includes heating to 53° C. to 67° C. Preferably, the second method includes heating to 54° C. to 65° C.

More details regarding the second method may be found in the description related to Embodiment 3.

In some embodiments, the third method includes heating the acequinocyl represented by formula (I) to 55° C. to 70° C. to melt, cooling to crystallize, and filtering to obtain the crystal form of the acequinocyl represented by formula (I).

Preferably, the third method includes heating to 56° C. to 69° C. Preferably, the third method includes heating to 57° C. to 68° C. Preferably, the third method includes heating to 58° C. to 67° C. Preferably, the third method includes heating to 59° C. to 66° C. Preferably, the third method includes heating to 60° C. to 65° C. Preferably, the third method includes heating to 61° C. to 64° C. Preferably, the third method includes heating to 62° C. to 63° C. Preferably, the third method includes heating to 60° C.

In some embodiments, a crystallization temperature for cooling crystallization in the third method may be the same as the crystallization temperature in the first method.

In some embodiments of the present disclosure, by using the first method, the second method, or the third method to prepare the crystal form of the acequinocyl represented by formula (I) can ensure that a desired crystal form of acequinocyl represented by formula (I) is obtained.

In some embodiments, when a reaction solvent for a synthesis reaction of the acequinocyl represented by formula (I) is the same as the crystallization solvent described in the embodiments of the present disclosure, the reaction solution containing the acequinocyl represented by formula (I) is processed by evaporating a portion of the reaction solvent, or used directly without any treatment, then dissolved in the crystallization solvent. The resulting solution is first heated to 55° C. to 70° C., then cooled to 0° C. to 60° C., followed by crystallization and filtration, to obtain the desired crystal form of the acequinocyl represented by formula (I). Any technical solutions identical or similar to this one also fall within the scope of inventive concepts of the embodiments of the present disclosure.

One or more embodiments of the present disclosure provide an agrochemical composition. The agrochemical composition includes the crystal form of the acequinocyl represented by formula (I) as described in the embodiments of the present disclosure and one or more excipients or additives.

The agrochemical composition refers to a mixture of various chemical components usable in agricultural production. For example, the agrochemical composition includes fertilizers, pesticides, or the like. The agrochemical composition may be in various forms. For example, the agrochemical composition is in a form such as a soluble liquid concentrate, an emulsifiable concentrate, a microemulsion, a suspension concentrate, a water-dispersible powder, or a granule, etc. In some embodiments, preferably, the agrochemical composition is in the form of the suspension concentrate. For example, the agrochemical composition is in the form of an aqueous suspension concentrate (i.e., the aforementioned suspension concentrate). In some embodiments, the agrochemical composition is a suspension concentrate of the acequinocyl represented by formula (I).

In some embodiments, the agrochemical composition includes the crystal form of the acequinocyl represented by formula (I) in an amount of about 1% to about 99% by weight of the agrochemical composition. In some embodiments, the agrochemical composition includes the crystal form of the acequinocyl represented by formula (I) in an amount of about 10% to about 90% by weight of the agrochemical composition. In some embodiments, the agrochemical composition includes the crystal form of the acequinocyl represented by formula (I) in an amount of about 20% to about 80% by weight of the agrochemical composition. In some embodiments, the agrochemical composition includes the crystal form of the acequinocyl represented by formula (I) in an amount of about 30% to about 70% by weight of the agrochemical composition. In some embodiments, the agrochemical composition includes the crystal form of the acequinocyl represented by formula (I) in an amount of about 40% to about 60% by weight of the agrochemical composition. In some embodiments, the agrochemical composition includes the crystal form of the acequinocyl represented by formula (I) in an amount of about 50% by weight of the agrochemical composition. It is understandable that the amount of the acequinocyl represented by formula (I) used in the agrochemical composition may be adjusted as appropriate based on a desired mite control effect, a formulation stability effect, etc.

The excipients refer to substances that assist in the formation of a formulation. The additives refer to auxiliary substances added during the processing or application of a formulation. In some embodiments, the excipients or the additives are selected from adjuvants or surfactants, including but not limited to wetting agents, emulsifiers, dispersants, viscosity modifiers, antifoaming agents, antifreeze agents, pH regulators, stabilizers, anti-caking agents, or the like.

In some embodiments, in addition to the crystal form of the acequinocyl represented by formula (I), the agrochemical composition further includes other active ingredients. In some embodiments, the other active ingredients are one or more other insecticides, acaricides, or the like. For example, the other active ingredients include organophosphorus compounds, carbamate compounds, pyrethroid compounds, or the like. In some embodiments, the other active ingredients are one or more herbicides or fungicides. For example, the other active ingredients include diphenyl ether compounds, triadimefon compounds, organic amine compounds, or the like.

One or more embodiments of the present disclosure provide a method for controlling agricultural pests. The method includes subjecting the agricultural pests or a region in which the agricultural pests reside to the crystal form of the acequinocyl represented by formula (I) as described in the embodiments of the present disclosure, or to the agrochemical composition as described in the embodiments of the present disclosure.

The crystal form of the acequinocyl represented by formula (I) and its preparation method described in the embodiments of the present disclosure may be elaborated in detail below with reference to Embodiments 1 to 4 and Comparative Example 1. It should be noted that reaction conditions, reaction materials and their dosages in Embodiments 1 to 4 and Comparative Example 1 are merely provided for the purpose of illustrating the crystal form of the acequinocyl represented by formula (I) and its preparation method, and do not limit the protection scope of the present disclosure.

### Detection Methods

An analytical instrument for the DSC is NETZSCH DCS214; measurement methods and parameters for the DSC are set as follows: a measurement temperature is raised from 0° C. to 150° C. at a heating rate of 5° C./min.

An instrument for the XRPD is PANalytical Empyrean; test methods and parameters for the XRPD are set as follows: the X-ray powder diffraction pattern is obtained with a copper radiation target at a scanning rate of 0.020 per minute.

### Embodiment 1

2.24 kg of crude acequinocyl represented by formula (I) (with a purity of 95%) was charged into a 10 L reaction kettle, followed by addition of 4 kg of methanol (i.e., the aforementioned crystallization solvent). The mixture was heated to 60° C., and after the acequinocyl represented by formula (I) was completely dissolved, the solution was slowly cooled to 5° C. to 10° C. (i.e., the aforementioned crystallization temperature), and the resulting mixture was filtered to obtain 2.11 kg of Product 1. Product 1 has a purity of 99% and a yield of 94%.

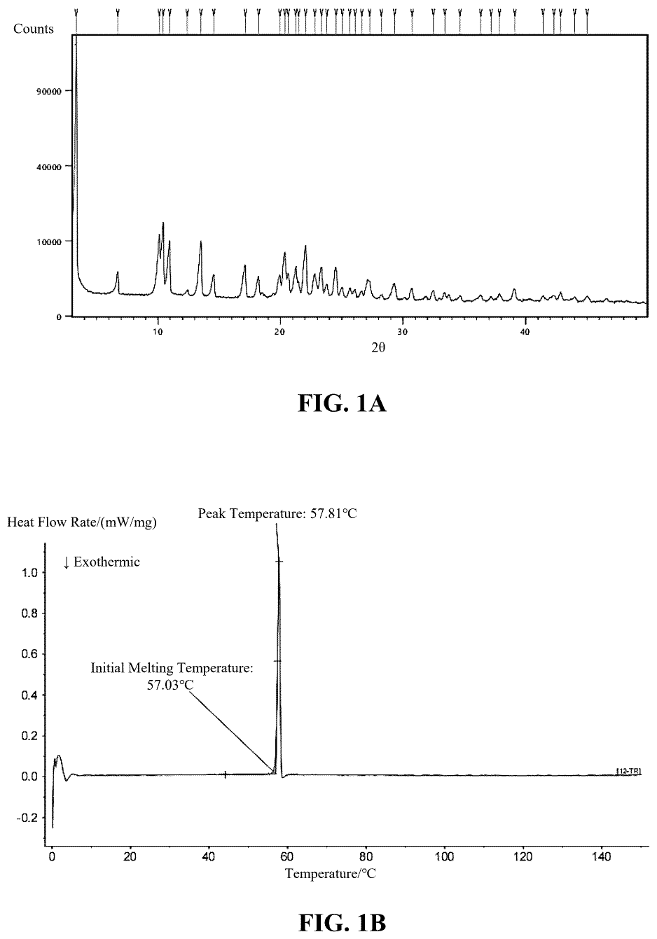

FIG. 1A is an X-ray powder diffraction pattern of a crystal form of acequinocyl represented by formula (I) prepared in Embodiment 1 of the present disclosure. As shown in FIG. 1A, the X-ray powder diffraction pattern of Product 1 has characteristic diffraction peaks at 3.3465° 2θ, 6.7245° 2θ, 10.4289° 2θ, 10.9607° 2θ, 13.5235° 2θ, 14.5662° 2θ, 17.1189° 2θ, 22.0684° 2θ, and 24.5351° 2θ.

FIG. 1B is a differential scanning calorimetry thermogram of the crystal form of acequinocyl represented by formula (I) prepared in Embodiment 1 of the present disclosure. As shown in FIG. 11B, the differential scanning calorimetry thermogram of Product 1 has a characteristic thermal event in the range of 57.03° C. to 57.81° C.

As shown in FIG. 1A and FIG. 11B, Product 1 prepared by the first method is the crystal form of the acequinocyl represented by formula (I) described in the foregoing embodiments.

Table 1 shows specific data of the X-ray powder diffraction pattern of Product 1 (including a diffraction angle, a d value, and a relative intensity of peaks).

TABLE 1
Data of the X-ray powder diffraction pattern of Product 1

| | Diffraction | | |
|---|---|---|---|
| No. | Angle [°2θ] | d Value [Å] | Relative Intensity [%] |
| 1 | 3.3465 | 26.40195 | 100.00 |
| 2 | 6.7245 | 13.14498 | 1.88 |
| 3 | 10.1118 | 8.74795 | 8.00 |
| 4 | 10.4289 | 8.48264 | 10.52 |
| 5 | 10.9607 | 8.07226 | 6.65 |
| 6 | 12.3997 | 7.13850 | 0.37 |
| 7 | 13.5235 | 6.54775 | 6.47 |
| 8 | 14.5662 | 6.08129 | 1.72 |
| 9 | 17.1189 | 5.17979 | 2.86 |
| 10 | 18.2095 | 4.87194 | 1.51 |
| 11 | 19.9447 | 4.45184 | 1.74 |
| 12 | 20.3628 | 4.36136 | 4.71 |
| 13 | 20.6406 | 4.30328 | 1.88 |
| 14 | 21.2517 | 4.18090 | 2.78 |
| 15 | 21.4764 | 4.13767 | 1.03 |
| 16 | 22.0684 | 4.02799 | 5.90 |
| 17 | 22.8025 | 3.89995 | 1.91 |
| 18 | 23.3549 | 3.80895 | 2.65 |
| 19 | 23.7872 | 3.74069 | 0.93 |
| 20 | 24.5351 | 3.62833 | 2.56 |
| 21 | 25.0474 | 3.55527 | 0.67 |
| 22 | 25.6608 | 3.47166 | 0.62 |
| 23 | 26.0729 | 3.41772 | 0.53 |
| 24 | 26.6359 | 3.34674 | 0.42 |
| 25 | 27.2813 | 3.26901 | 1.13 |
| 26 | 28.2134 | 3.16310 | 0.21 |
| 27 | 29.2971 | 3.04852 | 0.97 |
| 28 | 30.7214 | 2.91035 | 0.67 |
| 29 | 32.4752 | 2.75708 | 0.52 |
| 30 | 33.4168 | 2.68151 | 0.40 |
| 31 | 34.6353 | 2.58991 | 0.23 |
| 32 | 36.3180 | 2.47368 | 0.28 |
| 33 | 37.1624 | 2.41940 | 0.19 |
| 34 | 37.8671 | 2.37598 | 0.34 |
| 35 | 39.1047 | 2.30359 | 0.62 |
| 36 | 41.3969 | 2.18118 | 0.24 |
| 37 | 42.2979 | 2.13678 | 0.25 |
| 38 | 42.8397 | 2.11100 | 0.41 |
| 39 | 43.9987 | 2.05805 | 0.19 |
| 40 | 45.0214 | 2.01365 | 0.24 |

### Embodiment 2

2.24 kg of crude acequinocyl represented by formula (I) (with a purity of 950%) was charged into a 10 L reaction kettle, followed by addition of 4 kg of ethanol (i.e., the aforementioned crystallization solvent). The mixture was heated to 60° C., and after the acequinocyl represented by formula (I) was completely dissolved, the solution was slowly cooled to 5° C. to 10° C. (i.e., the aforementioned crystallization temperature), and the resulting mixture was filtered to obtain 2.11 kg of Product 2. Product 2 has a purity of 990% and a yield of 940%.

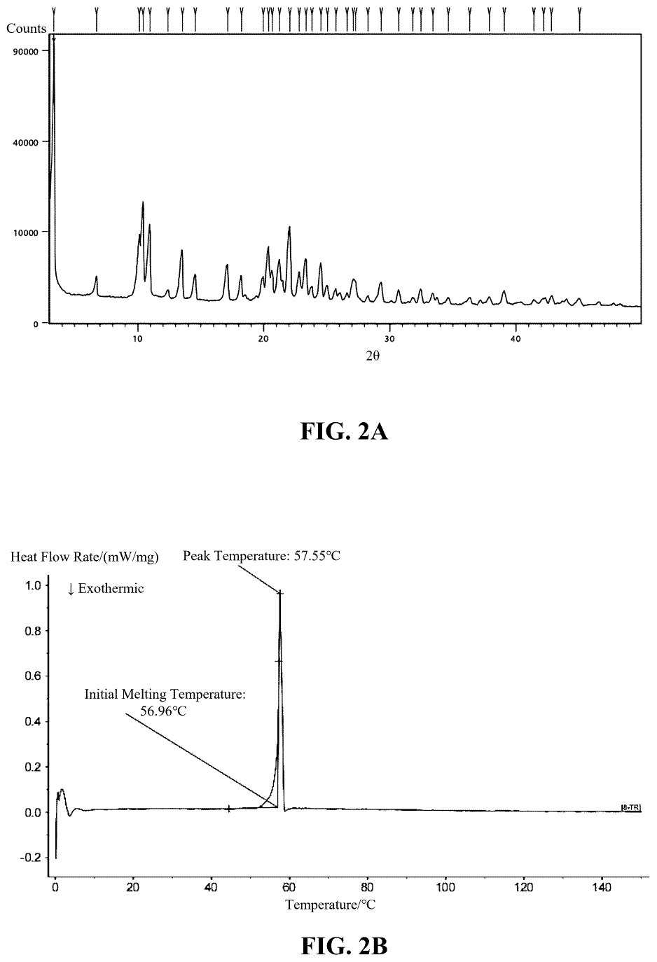

FIG. 2A is an X-ray powder diffraction pattern of a crystal form of acequinocyl represented by formula (I) prepared in Embodiment 2 of the present disclosure. As shown in FIG. 2A, the X-ray powder diffraction pattern of Product 2 has characteristic diffraction peaks at 3.3513° 2θ, 6.7269° 2θ, 10.4262° 2θ, 10.9626° 2θ, 13.5256° 2θ, 14.5699° 2θ, 17.1275° 2θ, 22.0641° 2θ, and 24.5299° 2θ.

FIG. 2B is a differential scanning calorimetry thermogram of the crystal form of acequinocyl represented by formula (I) prepared in Embodiment 2 of the present disclosure. As shown in FIG. 2B3, the differential scanning calorimetry thermogram of Product 2 has a characteristic thermal event in a range of 56.96° C. to 57.55° C.

As shown in FIG. 2A and FIG. 2B3, Product 2 prepared by the first method is the crystal form of the acequinocyl represented by formula (I) described in the foregoing embodiments.

Table 2 shows specific data of the X-ray powder diffraction pattern of Product 2.

TABLE 2
Data of the X-ray powder diffraction pattern of Product 2

| | Diffraction | | |
|---|---|---|---|
| No. | Angle [°2θ] | d Value [Å] | Relative Intensity [%] |
| 1 | 3.3513 | 26.36441 | 100.00 |
| 2 | 6.7269 | 13.14023 | 1.77 |
| 3 | 10.1144 | 8.74571 | 8.54 |
| 4 | 10.4262 | 8.48486 | 16.70 |
| 5 | 10.9626 | 8.07084 | 11.03 |
| 6 | 12.3807 | 7.14941 | 0.59 |
| 7 | 13.5256 | 6.54670 | 5.65 |
| 8 | 14.5699 | 6.07974 | 2.09 |
| 9 | 17.1275 | 5.17720 | 3.36 |
| 10 | 18.2108 | 4.87161 | 2.10 |
| 11 | 19.9398 | 4.45291 | 2.05 |
| 12 | 20.3659 | 4.36071 | 6.37 |
| 13 | 20.6553 | 4.30025 | 2.70 |
| 14 | 21.2520 | 4.18085 | 4.30 |
| 15 | 22.0641 | 4.02876 | 10.19 |
| 16 | 22.8056 | 3.89942 | 2.61 |
| 17 | 23.3557 | 3.80882 | 4.15 |
| 18 | 23.8138 | 3.73657 | 1.05 |
| 19 | 24.5299 | 3.62909 | 3.73 |
| 20 | 25.0285 | 3.55791 | 1.15 |
| 21 | 25.7120 | 3.46486 | 0.90 |
| 22 | 26.5817 | 3.35344 | 0.56 |
| 23 | 27.1043 | 3.28996 | 1.86 |
| 24 | 27.2735 | 3.26993 | 1.52 |
| 25 | 28.2400 | 3.16017 | 0.39 |
| 26 | 29.3027 | 3.04794 | 1.53 |
| 27 | 30.7030 | 2.91205 | 0.85 |
| 28 | 31.8255 | 2.81186 | 0.29 |
| 29 | 32.4576 | 2.75853 | 0.88 |
| 30 | 33.4248 | 2.68089 | 0.57 |
| 31 | 34.6344 | 2.58998 | 0.34 |
| 32 | 36.3431 | 2.47204 | 0.33 |
| 33 | 37.8710 | 2.37575 | 0.41 |
| 34 | 39.0760 | 2.30521 | 0.80 |
| 35 | 41.4040 | 2.18082 | 0.25 |
| 36 | 42.1965 | 2.14168 | 0.32 |
| 37 | 42.8122 | 2.11229 | 0.50 |
| 38 | 45.0253 | 2.01348 | 0.34 |

### Embodiment 3

2.24 kg of crude acequinocyl represented by formula (I) (with a purity of 99%) was charged into a 10 L reaction kettle and was heated to 60° C. to 70° C. After the crystal form of the acequinocyl represented by formula (I) was completely melted, and then was poured out of the reaction kettle, cooled and sliced to obtain 2.24 kg of Product 3. Product 3 has a purity of 99% and a yield of 100%.

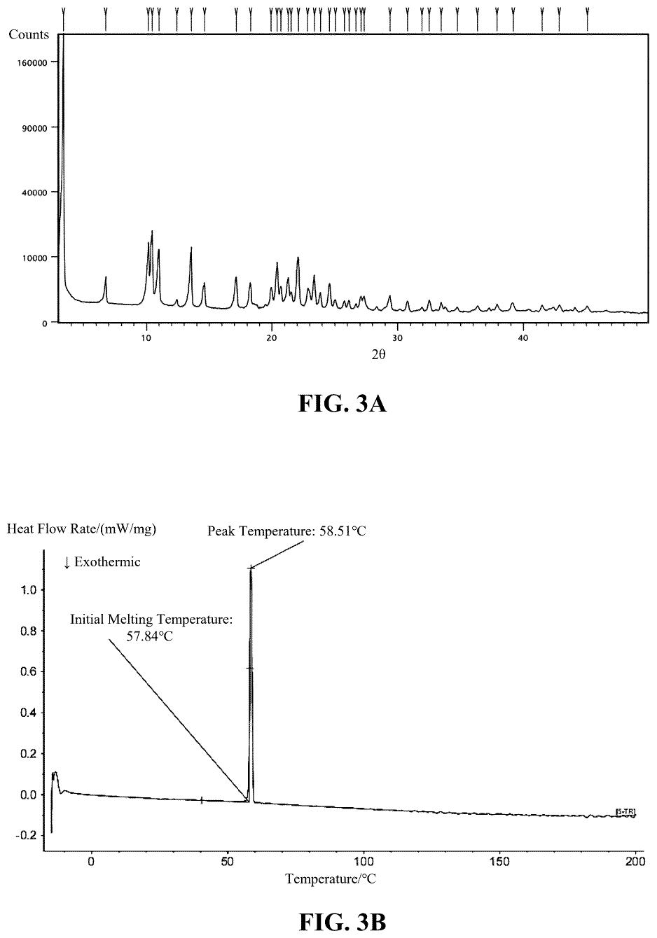

FIG. 3A is an X-ray powder diffraction pattern of a crystal form of acequinocyl represented by formula (I) prepared in Embodiment 3 of the present disclosure. As shown in FIG. 3A, the X-ray powder diffraction pattern of Product 3 has characteristic diffraction peaks at 3.3874° 2θ, 6.7684° 2θ, 10.4614° 2θ, 10.9971° 2θ, 13.5633° 2θ, 14.6231° 2θ, 17.1483° 2θ, 22.0848° 2θ, and 24.5728° 2θ.

FIG. 3B is a differential scanning calorimetry thermogram of the crystal form of acequinocyl represented by formula (I) prepared in Embodiment 3 of the present disclosure. As shown in FIG. 3B, the differential scanning calorimetry thermogram of Product 3 has a characteristic thermal event in a range of 57.84° C. to 58.51° C.

As shown in FIG. 3A and FIG. 3B, Product 3 prepared by the second method is the crystal form of the acequinocyl represented by formula (I) described in the foregoing embodiments.

Table 3 shows specific data of the X-ray powder diffraction pattern of Product 3.

TABLE 3
Data of the X-ray powder diffraction pattern of Product 3

| No. | Diffraction Angle [°2θ] | d Value [Å] | Relative Intensity [%] |
|---|---|---|---|
| 1 | 3.3874 | 26.08338 | 100.00 |
| 2 | 6.7684 | 13.05983 | 2.04 |
| 3 | 10.1553 | 8.71059 | 7.03 |
| 4 | 10.4614 | 8.45636 | 9.41 |
| 5 | 10.9971 | 8.04560 | 5.88 |
| 6 | 12.4359 | 7.11781 | 0.31 |
| 7 | 13.5633 | 6.52862 | 6.25 |
| 8 | 14.6231 | 6.05777 | 1.58 |
| 9 | 17.1483 | 5.17097 | 2.13 |
| 10 | 18.2857 | 4.85181 | 1.55 |
| 11 | 19.9180 | 4.45774 | 1.20 |
| 12 | 20.4066 | 4.35209 | 4.01 |
| 13 | 20.6900 | 4.29311 | 1.32 |
| 14 | 21.2807 | 4.17528 | 2.11 |
| 15 | 21.5225 | 4.12891 | 0.89 |
| 16 | 22.0848 | 4.02504 | 4.65 |
| 17 | 22.8362 | 3.89427 | 1.11 |
| 18 | 23.3647 | 3.80737 | 2.39 |
| 19 | 23.8499 | 3.73100 | 0.82 |
| 20 | 24.5728 | 3.62285 | 1.55 |
| 21 | 25.0297 | 3.55775 | 0.40 |
| 22 | 25.7626 | 3.45817 | 0.35 |
| 23 | 26.1119 | 3.41270 | 0.37 |
| 24 | 26.6598 | 3.34379 | 0.22 |
| 25 | 27.0594 | 3.29531 | 0.59 |
| 26 | 27.3130 | 3.26529 | 0.60 |
| 27 | 29.3712 | 3.04099 | 0.66 |
| 28 | 30.7562 | 2.90714 | 0.36 |
| 29 | 31.9208 | 2.80368 | 0.11 |
| 30 | 32.5008 | 2.75496 | 0.43 |
| 31 | 33.4429 | 2.67948 | 0.30 |
| 32 | 34.7271 | 2.58328 | 0.13 |
| 33 | 36.3328 | 2.47271 | 0.16 |
| 34 | 37.8858 | 2.37485 | 0.22 |
| 35 | 39.1900 | 2.29877 | 0.27 |
| 36 | 41.4681 | 2.17760 | 0.20 |
| 37 | 42.8238 | 2.11175 | 0.20 |
| 38 | 45.0703 | 2.01158 | 0.15 |

### Embodiment 4

2.24 kg of crude acequinocyl represented by formula (I) (with a purity of 950%) was charged into a 10 L reaction kettle, followed by addition of 24 kg of 2-methyltetrahydrofuran (i.e., the aforementioned crystallization solvent). The mixture was heated to 60° C., and after the acequinocyl represented by formula (I) was completely dissolved, the solution was slowly cooled to 15° C. to 20° C. (i.e., the aforementioned crystallization temperature), and the resulting mixture was filtered to obtain 2.11 kg of Product 4. Product 4 has a purity of 990% and a yield of 940%.

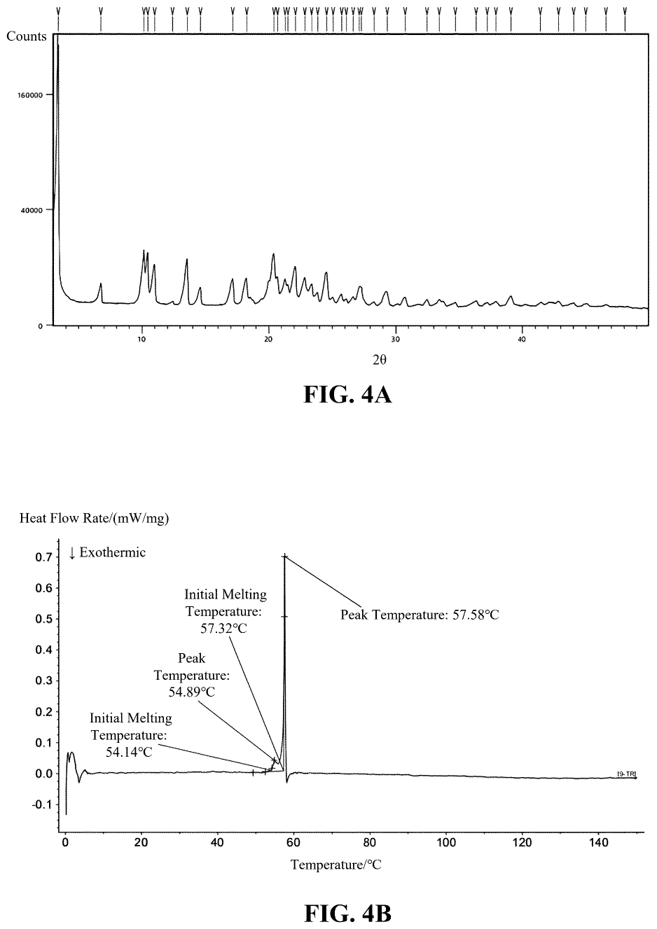

FIG. 4A is an X-ray powder diffraction pattern of a crystal form of acequinocyl represented by formula (I) prepared in Embodiment 4 of the present disclosure. As shown in FIG. 4A, the X-ray powder diffraction pattern of Product 4 has characteristic diffraction peaks at 3.3794° 2θ, 6.7564° 2θ, 10.4483° 2θ, 10.9867° 2θ, 13.5568° 2θ, 14.5973° 2θ, 17.1544° 2θ, 22.0885° 2θ, and 24.5680° 2θ.

FIG. 4B is a differential scanning calorimetry thermogram of the crystal form of acequinocyl represented by formula (I) prepared in Embodiment 4 of the present disclosure. As shown in FIG. 4B3, the differential scanning calorimetry thermogram of Product 4 has a characteristic thermal event in each of ranges of 54.14° C. to 54.89° C. and 57.32° C. to 57.58° C.

As shown in FIG. 4A and FIG. 4B3, Product 4 prepared by the first method is the crystal form of the acequinocyl represented by formula (I) described in the foregoing embodiments.

Table 4 shows specific data of the X-ray powder diffraction pattern of Product 4.

TABLE 4
Data of the X-ray powder diffraction pattern of Product 4

| No. | Diffraction Angle [°2θ] | d Value [Å] | Relative Intensity [%] |
|---|---|---|---|
| 1 | 3.3794 | 26.14517 | 100.00 |
| 2 | 6.7564 | 13.08295 | 1.50 |
| 3 | 10.1493 | 8.71570 | 6.18 |
| 4 | 10.4483 | 8.46696 | 5.62 |
| 5 | 10.9867 | 8.05324 | 3.81 |
| 6 | 12.4144 | 7.13008 | 0.17 |
| 7 | 13.5568 | 6.53171 | 4.67 |
| 8 | 14.5973 | 6.06839 | 1.20 |
| 9 | 17.1544 | 5.16915 | 1.99 |
| 10 | 18.2453 | 4.86246 | 2.05 |
| 11 | 20.3955 | 4.35445 | 5.54 |
| 12 | 20.6836 | 4.29443 | 2.25 |
| 13 | 21.2721 | 4.17694 | 2.04 |
| 14 | 21.5088 | 4.13151 | 1.49 |
| 15 | 22.0885 | 4.02437 | 3.49 |
| 16 | 22.8328 | 3.89485 | 2.24 |
| 17 | 23.3774 | 3.80532 | 1.53 |
| 18 | 23.8396 | 3.73258 | 0.80 |
| 19 | 24.5680 | 3.62355 | 2.74 |
| 20 | 25.0586 | 3.55370 | 0.46 |
| 21 | 25.7309 | 3.46236 | 0.70 |
| 22 | 26.1188 | 3.41181 | 0.38 |
| 23 | 26.6566 | 3.34419 | 0.49 |
| 24 | 27.1185 | 3.28826 | 1.26 |
| 25 | 27.3090 | 3.26576 | 1.22 |
| 26 | 28.2894 | 3.15477 | 0.22 |
| 27 | 29.3348 | 3.04468 | 0.83 |
| 28 | 30.7516 | 2.90757 | 0.49 |
| 29 | 32.4739 | 2.75718 | 0.36 |
| 30 | 33.4177 | 2.68144 | 0.36 |
| 31 | 34.7022 | 2.58508 | 0.21 |
| 32 | 36.3528 | 2.47140 | 0.30 |
| 33 | 37.2286 | 2.41524 | 0.21 |
| 34 | 37.8917 | 2.37450 | 0.29 |
| 35 | 39.0769 | 2.30516 | 0.64 |
| 36 | 41.4033 | 2.18086 | 0.22 |
| 37 | 42.8493 | 2.11055 | 0.28 |
| 38 | 44.0371 | 2.05634 | 0.22 |
| 39 | 44.9903 | 2.01497 | 0.18 |
| 40 | 46.5724 | 1.95014 | 0.15 |
| 41 | 48.0653 | 1.89300 | 0.06 |

### Comparative Example 1

Commercially available acequinocyl powder with a purity of 9600 was used in Comparative Example 1.

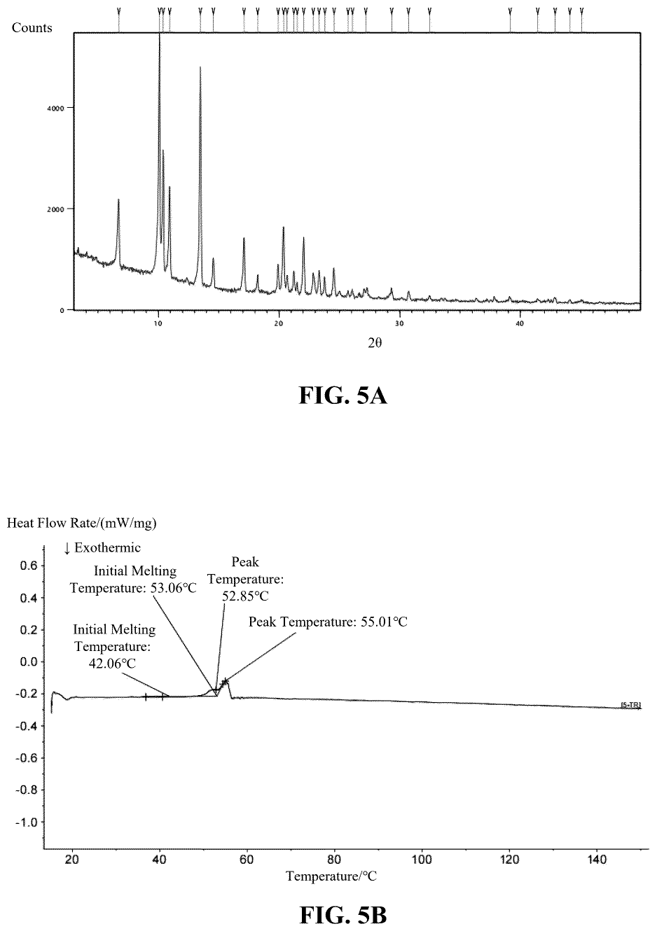

FIG. 5A is an X-ray powder diffraction pattern of a crystal form of acequinocyl in Comparative Example 1 of the present disclosure. As shown in FIG. 5A, characteristic diffraction peaks of the commercially available product of Comparative Example 1 are mainly distributed at 6.7123° 2θ, 10.0932° 2θ, 10.4008° 2θ, 10.9359° 2θ, 13.4920° 2θ, 17.1026° 2θ, 20.3673° 2θ, 22.0386° 2θ, etc.

FIG. 5B is a differential scanning calorimetry thermogram of the crystal form of acequinocyl in Comparative Example 1 of the present disclosure. As shown in FIG. 5B3, the commercially available product of Comparative Example 1 has a characteristic thermal event in each of ranges of 42.06° C. to 52.85° C. and 53.06° C. to 55.01° C.

As shown in FIG. 5A and FIG. 5B3, the commercially available product is different from the crystal form of the acequinocyl represented by formula (I) described in the foregoing embodiments.

Table 5 shows specific data of the X-ray powder diffraction pattern of the commercially available product of Comparative Example 1.

TABLE 5
Data of the X-ray powder diffraction pattern of the commercially available product

| No. | Diffraction Angle [°2θ] | d Value [Å] | Relative Intensity [%] |
|---|---|---|---|
| 1 | 6.7123 | 13.16880 | 27.83 |
| 2 | 10.0932 | 8.76403 | 100.00 |
| 3 | 10.4008 | 8.50552 | 52.15 |
| 4 | 10.9359 | 8.09050 | 37.70 |
| 5 | 13.4920 | 6.56295 | 88.31 |
| 6 | 14.5470 | 6.08927 | 12.28 |
| 7 | 17.1026 | 5.18469 | 21.84 |
| 8 | 18.2350 | 4.86520 | 7.44 |
| 9 | 19.9261 | 4.45596 | 11.94 |
| 10 | 20.3673 | 4.36041 | 27.49 |
| 11 | 20.6709 | 4.29705 | 7.75 |
| 12 | 21.2352 | 4.18411 | 9.36 |
| 13 | 21.4974 | 4.13367 | 4.81 |
| 14 | 22.0386 | 4.03337 | 23.57 |
| 15 | 22.8363 | 3.89426 | 9.08 |
| 16 | 23.3214 | 3.81434 | 10.17 |
| 17 | 23.7825 | 3.74142 | 7.60 |
| 18 | 24.5500 | 3.62616 | 11.39 |
| 19 | 25.7120 | 3.46486 | 2.14 |
| 20 | 26.0736 | 3.41763 | 2.71 |
| 21 | 27.1805 | 3.28091 | 2.84 |
| 22 | 29.3303 | 3.04514 | 4.03 |
| 23 | 30.7235 | 2.91016 | 3.47 |
| 24 | 32.4595 | 2.75837 | 1.43 |
| 25 | 39.1316 | 2.30207 | 1.77 |
| 26 | 41.4336 | 2.17933 | 1.09 |
| 27 | 42.8574 | 2.11017 | 1.95 |
| 28 | 44.0878 | 2.05410 | 1.27 |
| 29 | 45.0703 | 2.01157 | 0.80 |

### Determination and Result Analysis of Formulation Stability

Preparation of aqueous suspension concentrates: Acequinocyl aqueous suspension concentrates are prepared separately from the crystal form of the acequinocyl represented by formula (I) described in the embodiments of the present disclosure (i.e., Products 1 to 4 in Embodiments 1 to 4) and commercially available acequinocyl (i.e., the commercially available acequinocyl in Comparative Example 1). A specific method is as follows: 10 wt % acequinocyl, 6.0 wt % comblike grafted acrylic acid copolymer Atlox 4917 (CRODA), 0.3 wt % xanthan gum, 0.5 wt % magnesium aluminum silicate, 0.5 wt % isothiazolinone, 5 wt % ethylene glycol, 0.5 wt dimethyl silicone oil, and water (the balance) are mixed. The mixture is then shear-mixed for a certain period of time, and the resulting slurry is sand-milled to a particle size of less than 5 micrometers to obtain an aqueous suspension concentrate with a uniform appearance and good dispersibility in water.

Thermal storage stability test: the thermal storage stability test is performed on the prepared aqueous suspension concentrates, involving thermal storage at 54° C. for 14 days (conducted in accordance with GB/T 19136-2021, the Chinese National Standard Determination Method for Thermal Storage Stability of Pesticides).

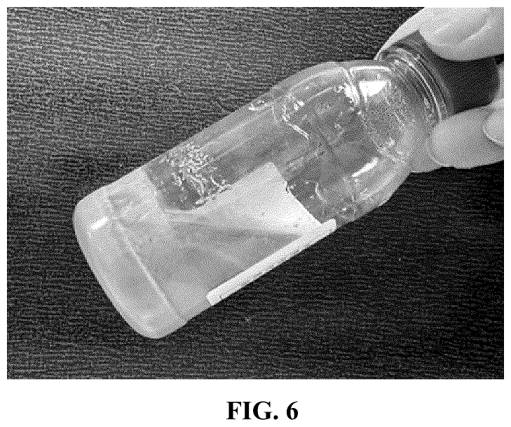

FIG. 6 is a photograph illustrating stability of Comparative Example 1 of the present disclosure after thermal storage followed by storage at room temperature for 2 hours. As shown in FIG. 6, the aqueous suspension concentrate prepared from the commercially available acequinocyl (i.e., the commercially available acequinocyl in Comparative Example 1) demonstrates a significant creaming phenomenon and poor fluidity after thermal storage followed by storage at room temperature for 2 hours.

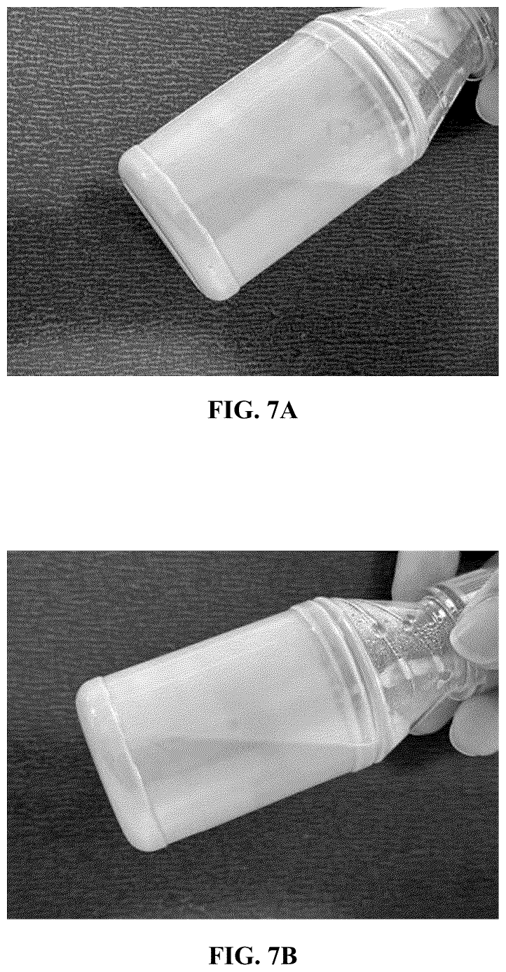

FIG. 7A is a photograph illustrating stability of the crystal form of acequinocyl represented by formula (I) of Embodiment 1 of the present disclosure after thermal storage followed by storage at room temperature for 2 hours. As shown in FIG. 7A, the aqueous suspension concentrate prepared from the crystal form of the acequinocyl represented by formula (I) (i.e., Product 1 of Embodiment 1) described in the embodiments of the present disclosure demonstrates a normal appearance, no creaming phenomenon, and good fluidity after thermal storage followed by storage at room temperature for 2 hours.

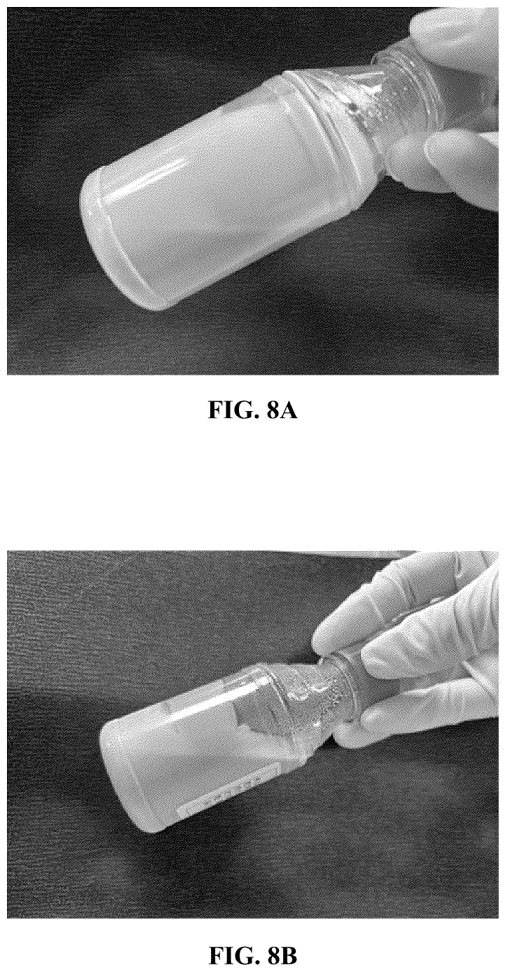

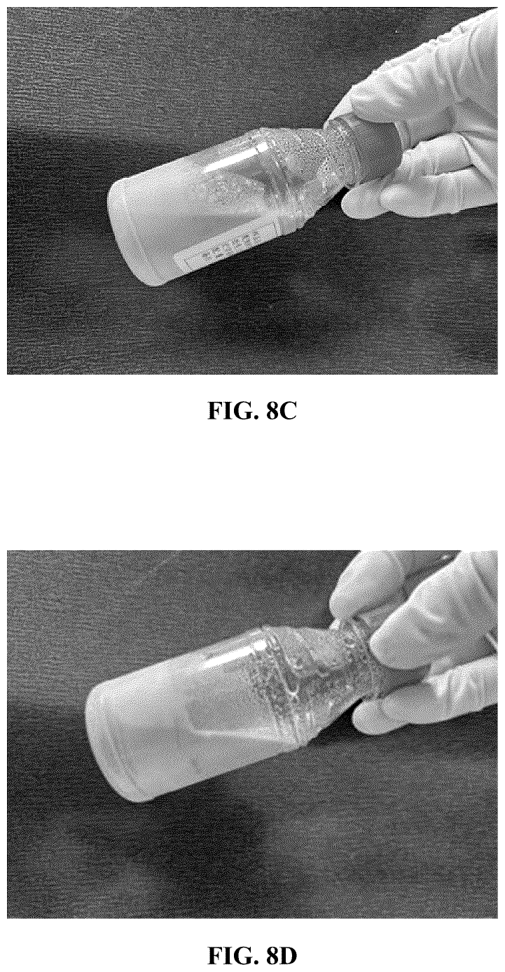

To further observe whether the aqueous suspension concentrate prepared from the crystal form of acequinocyl represented by formula (I) described in the embodiments of the present disclosure can adapt to extremely harsh environmental conditions, after thermal storage at 54° C. for 14 days, the aqueous suspension concentrate is stored at room temperature for 14 days and 1 month, respectively. FIG. 7B is a photograph illustrating stability of the crystal form of acequinocyl represented by formula (I) of Embodiment 1 of the present disclosure after thermal storage followed by storage at room temperature for 14 days. As shown in FIG. 7B, the aqueous suspension concentrate prepared from Product 1 of Embodiment 1 maintains a normal appearance and good fluidity with no obvious creaming after 14 days. FIGS. 8A to 8D are photographs illustrating stability of the crystal forms of acequinocyl represented by formula (I) of Embodiments 1 to 4 of the present disclosure after thermal storage followed by storage at room temperature for 1 month, respectively. As shown in FIGS. 8A to 8D, the aqueous suspension concentrates prepared from Products 2-4 of Embodiments 2 to 4 show no creaming or minor crystallization with good fluidity after 1 month. Therefore, the aqueous suspension concentrates prepared from Products 2-4 of Embodiments 1 to 4 exhibit excellent thermal storage stability. Among them, Product 1 of Embodiment 1 exhibits particularly excellent performance with the best stability, followed by Product 2 of Embodiment 2 and Product 3 of Embodiment 3, and Product 4 of Embodiment 4 has the lowest stability.

To further quantify the effects of different acequinocyl crystal forms on stabilities of the prepared acequinocyl aqueous suspension concentrates, a particle size test is conducted. The test adopts a laser particle size analyzer method, using a BT-9300S laser particle size analyzer to determine particle sizes of the acequinocyl aqueous suspension concentrates prepared from products of the aforementioned Comparative Example 1 and Embodiments 1 to 4.

Table 6 shows specific data of the particle sizes of the acequinocyl aqueous suspension concentrates prepared from products of the Comparative Example 1 and Embodiments 1 to 4.

TABLE 6
data of the particle sizes of the acequinocyl aqueous suspension concentrates
prepared from products of the Comparative Example 1 and Embodiments 1 to 4

| | | Particle sizes | | Particle sizes | | Particle sizes | |
|---|---|---|---|---|---|---|---|
| | Particle | after thermal | | after thermal | | after thermal | |
| | sizes | storage at 54° C. | | storage at 54° C. | | storage at 54° C. | |
| Acequinocyl | before | followed by | Particle | followed by | Particle | followed by | Particle |
| aqueous | thermal | storage at room | size | storage at room | size | storage at room | size |
| suspension | storage | temperature for | increase/ | temperature for | increase/ | temperature for | increase/ |
| concentrates | (d90)/μm | 2 hours (d90)/μm | % | 14 days (d90)/μm | % | 30 days (d90)/μm | % |
| Comparative | 4.943 | 37.11 | 650.8% | / | / | / | / |
| example 1 | | | | | | | |
| Embodiment 1 | 4.469 | 6.653 | 48.9% | 7.762 | 73.7% | 7.873 | 76.2% |
| Embodiment 2 | 4.581 | 7.364 | 60.8% | 9.292 | 102.8% | 12.23 | 167.0% |
| Embodiment 3 | 4.803 | 7.961 | 65.8% | 9.188 | 91.3% | 13.84 | 188.2% |
| Embodiment 4 | 4.437 | 10.26 | 131.2% | 13.15 | 196.4% | 16.15 | 264.0% |

As shown in Table 6, for the acequinocyl aqueous suspension concentrate prepared from the commercially available acequinocyl in Comparative Example 1, its particle size increases sharply from 4.943 μm to 37.110 μm after thermal storage at 54° C. followed by storage at room temperature for 2 hours, with a particle size increase of 650.8%. This indicates that the acequinocyl aqueous suspension concentrate prepared from the commercially available acequinocyl in Comparative Example 1 undergoes rapid particle agglomeration and a sharp increase in particle size under thermal storage conditions, directly leading to a subsequent creaming risk and exhibiting poor stability. For the acequinocyl aqueous suspension concentrate prepared from Product 1 of Embodiment 1, its particle size increase are 48.9%, 73.7%, and 76.2%, respectively, after thermal storage at 54° C. followed by storage at room temperature for 2 hours, 14 days, and 30 days. The increase remains moderate and stable throughout the entire test period, showing optimal stability and a low creaming risk. The stability performances of the acequinocyl aqueous suspension concentrate prepared from Products 2-4 of Embodiments 2 to 4 are slightly inferior to that of Embodiment 1, but they are much better than that of Comparative Example 1. For the acequinocyl aqueous suspension concentrate prepared from Product 2 of Embodiment 2, its particle size increase are 60.8%, 102.8%, and 167%, respectively, after thermal storage at 54° C. followed by storage at room temperature for 2 hours, 14 days, and 30 days. For the acequinocyl aqueous suspension concentrate prepared from Product 3 of Embodiment 3, its particle size increase are 65.8%, 91.3%, and 188.2%, respectively, after thermal storage at 54° C. followed by storage at room temperature for 2 hours, 14 days, and 30 days. For the acequinocyl aqueous suspension concentrate prepared from Product 4 of Embodiment 4, its particle size increase are 131.2%, 196.4%, and 264.0%, respectively, after thermal storage at 54° C. followed by storage at room temperature for 2 hours, 14 days, and 30 days. Although the acequinocyl aqueous suspension concentrate prepared from Product 4 of Embodiment 4 shows a large increase relative to Embodiments 1-3, its particle size increase is still controlled within 300%.

Result analysis: The crystal form of the acequinocyl represented by formula (I) of the present disclosure exhibits excellent adaptability to an aqueous suspension concentrate system. The acequinocyl aqueous suspension concentrate prepared from this crystal form effectively solves the technical problem that the aqueous suspension concentrate prepared from the commercially available acequinocyl is prone to creaming and poor fluidity after thermal storage, and thus fails to meet technical requirements for production, transportation, and storage. The aqueous suspension concentrate prepared from this crystal form exhibits stability, fluidity, and anti-deterioration performance that are significantly superior to those of the aqueous suspension concentrate prepared from the commercially available acequinocyl under both room temperature and high-temperature conditions. The aqueous suspension concentrates prepared from Products 1-4 of Embodiments 1 to 4 all meet the full-process technical requirements for production, transportation, and storage.

The acequinocyl aqueous suspension concentrates prepared from Products 1-4 of Embodiments 1 to 4 of the present disclosure all exhibit excellent thermal storage stability, with the performance of each embodiment exhibiting a graded advantage: Embodiment 1 is the optimal solution. The aqueous suspension concentrate of the crystal form of the acequinocyl represented by formula (I), prepared using methanol as the crystallization solvent, maintains the optimal level of stability and fluidity throughout the full-period tests of short-term, medium-to-long-term thermal storage, and room temperature storage. Embodiments 2 and 3 follow, exhibiting a balanced performance in anti-crystallization, anti-creaming, and fluidity retention. Although Embodiment 4 ranks the lowest in performance among Embodiments 1 to 4, its overall performance is still significantly better than that of existing commercially available products, with no creaming risk, and its fluidity can meet the technical requirements for production, transportation, and storage.

In summary, the crystal form of the acequinocyl represented by formula (I) of the present disclosure is well adapted to the aqueous suspension concentrate system. The acequinocyl aqueous suspension concentrate prepared from this crystal form exhibits remarkable stability, uniformity, and good fluidity under both room temperature and elevated temperature conditions, thus effectively overcoming the technical defects of easy creaming and poor fluidity in the aqueous suspension concentrate prepared from the commercially available acequinocyl. Among them, the formulation system of Embodiment 1, prepared using methanol as the crystallization solvent, exhibits the optimal stability. Products prepared in Embodiments 2 to 4 can also stably meet the stringent technical requirements for production, transportation, and storage of agrochemical formulations. The present disclosure provides reliable performance support for the application of the crystal form of the acequinocyl represented by formula (I) in agrochemical acequinocyl aqueous suspension concentrates, and thus endows this crystal form with remarkable industrial application value.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended for those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this disclosure are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A crystal form of acequinocyl represented by formula (I), 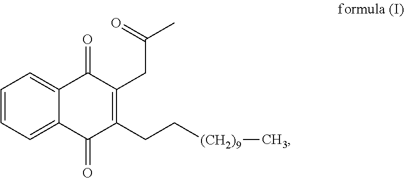
wherein an X-ray powder diffraction pattern (Cu-Kα radiation) of the crystal form has a characteristic diffraction peak at 3.3° 2θ±0.2° 2θ and a characteristic diffraction peak at at least one of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ.

2. The crystal form of claim 1, wherein the X-ray powder diffraction pattern (Cu-Kα radiation) of the crystal form has characteristic diffraction peaks at at least two of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ.

3. The crystal form of claim 2, wherein the X-ray powder diffraction pattern (Cu-Kα radiation) of the crystal form has characteristic diffraction peaks at at least three of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ.

4. The crystal form of claim 3, wherein the X-ray powder diffraction pattern (Cu-Kα radiation) of the crystal form has characteristic diffraction peaks at at least five of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ.

5. The crystal form of claim 4, wherein the X-ray powder diffraction pattern (Cu-Kα radiation) of the crystal form has characteristic diffraction peaks at at least seven of 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, or 24.5° 2θ±0.2° 2θ.

6. The crystal form of claim 5, wherein the X-ray powder diffraction pattern (Cu-Kα radiation) of the crystal form has characteristic diffraction peaks at 6.7° 2θ±0.2° 2θ, 10.1° 2θ±0.2° 2θ, 10.4° 2θ±0.2° 2θ, 11.0° 2θ±0.2° 2θ, 13.5° 2θ±0.2° 2θ, 14.6° 2θ±0.2° 2θ, 17.1° 2θ±0.2° 2θ, 22.1° 2θ±0.2° 2θ, and 24.5° 2θ±0.2° 2θ.

7. The crystal form of claim 6, wherein a relative intensity of the characteristic diffraction peak at 3.3° 2θ±0.2° 2θ is defined as 100%, and relative intensities of characteristic diffraction peaks at 10.1° 2θ±0.2° 2θ and 10.4° 2θ±0.2° 2θ are greater than 5%.

8. The crystal form of claim 7, wherein relative intensities of characteristic diffraction peaks at 11.0° 2θ±0.2° 2θ, 13.5° 2θ±0.2° 2θ, and 22.1° 2θ±0.2° 2θ are greater than 3%.

9. The crystal form of claim 8, wherein relative intensities of characteristic diffraction peaks at 6.7° 2θ±0.2° 2θ, 14.6° 2θ±0.2° 2θ, 17.1° 2θ±0.2° 2θ, and 24.5° 2θ±0.2° 2θ are greater than 1%.

10. The crystal form of claim 1, wherein
the X-ray powder diffraction pattern (Cu-Kα radiation) of the crystal form has characteristic diffraction peaks at 3.3465° 2θ±0.2° 2θ, 6.7245° 2θ±0.2° 2θ, 10.1118° 2θ±0.2° 2θ, 10.4289° 2θ±0.2° 2θ, 10.9607° 2θ±0.2° 2θ, 13.5235° 2θ±0.2° 2θ, 14.5662° 2θ±0.2° 2θ, 17.1189° 2θ±0.2° 2θ, 22.0684° 2θ±0.2° 2θ, and 24.5351° 2θ±0.2° 2θ;
the X-ray powder diffraction pattern (Cu-Kα radiation) of the crystal form has characteristic diffraction peaks at 3.3513° 2θ±0.2° 2θ, 6.7269° 2θ±0.2° 2θ, 10.1144° 2θ±0.2° 2θ, 10.4262° 2θ±0.2° 2θ, 10.9626° 2θ±0.2° 2θ, 13.5256° 2θ±0.2° 2θ, 14.5699° 2θ±0.2° 2θ, 17.1275° 2θ±0.2° 2θ, 22.0641° 2θ±0.2° 2θ, and 24.5299° 2θ±0.2° 2θ;
the X-ray powder diffraction pattern (Cu-Kα radiation) of the crystal form has characteristic diffraction peaks at 3.3874° 2θ±0.2° 2θ, 6.7684° 2θ±0.2° 2θ, 10.1553° 2θ±0.2° 2θ, 10.4614° 2θ±0.2° 2θ, 10.9971° 2θ±0.2° 2θ, 13.5633° 2θ±0.2° 2θ, 14.6231° 2θ±0.2° 2θ, 17.1483° 2θ±0.2° 2θ, 22.0848° 2θ±0.2° 2θ, and 24.5728° 2θ±0.2° 2θ; or
the X-ray powder diffraction pattern (Cu-Kα radiation) of the crystal form has characteristic diffraction peaks at 3.3794° 2θ±0.2° 2θ, 6.7564° 2θ±0.2° 2θ, 10.1493° 2θ±0.2° 2θ, 10.4483° 2θ±0.2° 2θ, 10.9867° 2θ±0.2° 2θ, 13.5568° 2θ±0.2° 2θ, 14.5973° 2θ±0.2° 2θ, 17.1544° 2θ±0.2° 2θ, 22.0885° 2θ±0.2° 2θ, and 24.5680° 2θ±0.2° 2θ.

11. The crystal form of claim 10, wherein
a relative intensity of the characteristic diffraction peak at 3.3465° 2θ±0.2° 2θ is defined as 100%, and relative intensities of the characteristic diffraction peaks at 6.7245° 2θ±0.2° 2θ, 10.1118° 2θ±0.2° 2θ, 10.4289° 2θ±0.2° 2θ, 10.9607° 2θ±0.2° 2θ, 13.5235° 2θ±0.2° 2θ, 14.5662° 2θ±0.2° 2θ, 17.1189° 2θ±0.2° 2θ, 22.0684° 2θ±0.2° 2θ, and 24.5351° 2θ±0.2° 2θ are 1.88%, 8%, 10.52%, 6.65%, 6.47%, 1.72%, 2.86%, 5.9%, and 2.56%, respectively;
a relative intensity of the characteristic diffraction peak at 3.3513° 2θ±0.2° 2θ is defined as 100%, and relative intensities of the characteristic diffraction peaks at 6.7269° 2θ±0.2° 2θ, 10.1144° 2θ±0.2° 2θ, 10.4262° 2θ±0.2° 2θ, 10.9626° 2θ±0.2° 2θ, 13.5256° 2θ±0.2° 2θ, 14.5699° 2θ±0.2° 2θ, 17.1275° 2θ±0.2° 2θ, 22.0641° 2θ±0.2° 2θ, and 24.5299° 2θ±0.2° 2θ are 1.77%, 8.54%, 16.7%, 11.03%, 5.65%, 2.09%, 3.36%, 10.19%, and 3.73%, respectively;
a relative intensity of the characteristic diffraction peak at 3.3874° 2θ±0.2° 2θ is defined as 100%, and relative intensities of the characteristic diffraction peaks at 6.7684° 2θ±0.2° 2θ, 10.1553° 2θ±0.2° 2θ, 10.4614° 2θ±0.2° 2θ, 10.9971° 2θ±0.2° 2θ, 13.5633° 2θ±0.2° 2θ, 14.6231° 2θ±0.2° 2θ, 17.1483° 2θ±0.2° 2θ, 22.0848° 2θ±0.2° 2θ, and 24.5728° 2θ±0.2° 2θ are 2.04%, 7.03%, 9.41%, 5.88%, 6.25%, 1.58%, 2.13%, 4.65%, and 1.55%, respectively; or
a relative intensity of the characteristic diffraction peak at 3.3794° 2θ±0.2° 2θ is defined as 100%, and relative intensities of the characteristic diffraction peaks at 6.7564° 2θ±0.2° 2θ, 10.1493° 2θ±0.2° 2θ, 10.4483° 2θ±0.2° 2θ, 10.9867° 2θ±0.2° 2θ, 13.5568° 2θ±0.2° 2θ, 14.5973° 2θ±0.2° 2θ, 17.1544° 2θ±0.2° 2θ, 22.0885° 2θ±0.2° 2θ, and 24.5680° 2θ±0.2° 2θ are 1.5%, 6.18%, 5.62%, 3.81%, 4.67%, 1.2%, 1.99%, 3.49%, and 2.74%, respectively.

12. The crystal form of claim 1, wherein a differential scanning calorimetry thermogram of the crystal form, obtained at a heating rate of 5° C./min, has at least one characteristic thermal event in a range of 55° C. to 60° C.

13. The crystal form of claim 12, wherein the differential scanning calorimetry thermogram of the crystal form has at least one characteristic thermal event in a range of 57° C. to 58° C.

14. The crystal form of claim 12, wherein the differential scanning calorimetry thermogram of the crystal form has at least one characteristic thermal event in a range of 58° C. to 59° C.

15. The crystal form of claim 12, wherein the differential scanning calorimetry thermogram of the crystal form has at least one characteristic thermal event in a range of 57.03° C. to 57.81° C., 56.96° C. to 57.55° C., 57.84° C. to 58.51° C., or 57.32° C. to 57.58° C.

16. The crystal form of claim 12, wherein an aqueous suspension concentrate prepared from the crystal form has a particle size increase of less than 300% after thermal storage at a temperature greater than 50° C. for over 10 days and subsequent storage at room temperature for not less than 30 days.

17. A method for preparing the crystal form of claim 1, comprising:
a first method including dissolving the acequinocyl represented by the formula (I) in a crystallization solvent, heating to 55° C. to 70° C., cooling to crystallize, and filtering to obtain the crystal form;
a second method including heating the acequinocyl represented by the formula (I) to 55° C. to 70° C. to melt, cooling and slicing to obtain the crystal form; or
a third method including heating the acequinocyl represented by the formula (I) to 55° C. to 70° C. to melt, cooling to crystallize, and filtering to obtain the crystal form.

18. The method of claim 17, wherein
in the first method, a weight ratio of the crystallization solvent to the acequinocyl represented by the formula (I) is (0.5-10):1;
in the first method and the third method, a crystallization temperature of the acequinocyl represented by the formula (I) is in a range of 0° C. to 70° C.; or
in the first method, the crystallization solvent includes at least one of water, alcohol, ether, or hydrocarbon.

19. The method of claim 18, wherein,
in the first method, the weight ratio of the crystallization solvent to the acequinocyl represented by the formula (I) is (1-5):1;
in the first method and the third method, the crystallization temperature of the acequinocyl represented by the formula (I) is in a range of 5° C. to 20° C.; or
the alcohol includes at least one of methanol, ethanol, or isopropanol, the ether includes 2-methyltetrahydrofuran, and the hydrocarbon includes at least one of n-hexane, cyclohexane, n-pentane, or n-heptane.

20. An agrochemical composition, comprising the crystal form of claim 1 and one or more excipients or additives.
